(19) United States
(12) Patent Application Publication
(10) Pub. No.: US 2022/0023533 A1
(43) Pub. Date: Jan. 27, 2022

(54) SUBCUTANEOUS DELIVERY OF HIGH CONCENTRATION FORMULATIONS

(71) Applicants: JAZZ PHRMACETICALS IRELAND LIMITED, Dublin (IE); ENABLE INJECTIONS, INC., Cincinnati, OH (US)

(72) Inventors: Mariana DIMITROVA, Wallington, PA (US); Matthew J. HUDDLESTON, Loveland, OH (US); Ql WANG, Pennington, NJ (US)

(21) Appl. No.: 17/311,575

(22) PCT Filed: Dec. 6, 2019
(86) PCT No.: PCT/US2019/064901

§ 371 Date: Jun. 7, 2021

Related U.S. Application Data
(60) Provisional application No. 62/776,500, filed on Dec. 7, 2018.

(51) Int. Cl. A61M 5/152; A61K 31/711; A61K 47/12; A61M 5/142; A61M 5/162

(52) U.S. Cl. CPC A61M 5/152; A61K 31/711; A61M 2005/14252; A61M 5/14248; A61M 5/162; A61K 47/12

(57) ABSTRACT

The present disclosure provides high concentration low viscosity defibrotide formulations administered via an automated injection device. The device is calibrated for optimum flow rate and administration of the high concentration low viscosity formulation.

### CROSS REFERENCE TO RELATED APPLICATIONS

This application claims priority under 35 U.S.C. § 119(e) to U.S. Provisional Application Ser. No. 62/776,500, filed Dec. 7, 2018, which is herein incorporated by reference in its entirety.

### BACKGROUND OF THE INVENTION

Defibrotide, a nucleic acid salt, is a complex mixture of random sequence, predominantly single-stranded polydeoxyribonucleotides derived from animal mucosal DNA. It has protective effects on vascular endothelial cells, particularly those of small vessels and has antithrombotic, anti-inflammatory and antiischemic properties.

The sodium salt of defibrotide is commercially sold as Defitelio® (Gentium S.r.L., Villa Guardia, Italy) and is currently approved for the treatment of adult and pediatric patients with hepatic veno-occlusive disease (VOD), also known as sinusoidal obstruction syndrome (SOS), with renal or pulmonary dysfunction following hematopoietic stem-cell transplantation (HSCT). It is administered to patients by 2-hour intravenous infusions every 6 hours for a minimum of 21 days. The frequency and large volumes of the infusion regimen requires that patients have a second IV line for defibrotide administration to avoid mixing defibrotide with other drugs that must be given IV.

Given the need for frequent IV administration, this treatment regimen is inconvenient, expensive (requiring a hospital stay), and is not compatible in an outpatient dosing for additional disease indications for which defibrotide may be shown to be effective in the prevention and/or treatment of rare orphan diseases.

### SUMMARY OF THE INVENTION

Described herein are injection devices and methods for convenient dosing of high concentration low-viscosity defibrotide formulations. Specifically, the injection devices and methods may be useful when the subcutaneous (SC) route of administration of the formulations is desired. The SC route of administration may offer an improved quality of life for the patients undergoing treatment with the defibrotide formulations, as further described below. The injection devices may also be useful for dose preparation by the patient prior to administration. The injection devices may be an automated, disposable, wearable, and one-time-use device and configured to allow the patient to self-administer the formulations.

The present disclosure also provides methods of preparing and/or administering a high concentration defibrotide formulation to a patient via an automated injection device, wherein the automated injection device generally includes: a) a housing defining a central portion, wherein the housing has a circular profile and includes a spiral recess; b) a bladder positioned within the housing, the bladder including a first end, a second end, and a wall extending between the first end and the second end, the bladder being expandable, wherein the bladder wall includes an area of increased thickness along a side of the bladder wall, where the area of increased thickness deflects less than a remaining portion of the bladder wall during administration to effect movement of the distal end of the bladder along an arcuate path, resulting in constant pressure-driven dose delivery; and c) a cannula disposed within the central portion of the housing and configured to be movable between multiple positions, including a pre-dispense position where the cannula is not in fluid communication with the bladder, and a dispense position where the cannula is in fluid flow communication with the bladder. The cannula may have an inner diameter that ranges from about 0.08 mm to about 0.41 mm, or from about 0.1 mm to about 0.3 mm.

The high concentration defibrotide formulations delivered by the disclosed methods generally comprise between 100 mg/mL to about 400 mg/mL of defibrotide, and a viscosity reducer at a concentration of between about 5 mM and about 100 mM. In some embodiments, the viscosity reducer is glycylglycine, glycine, sodium citrate, or benzyl alcohol. These high concentration defibrotide formulations may be formulated for parenteral delivery, e.g., subcutaneous delivery, to a patient, and may have a viscosity between about 5 and about 70 cP when measured at a temperature between 15° C. and 25° C., and an osmolality between about 240 mOsm/kg and about 1000 mOsm/kg.

Delivery, preparation and/or administration of the high concentration defibrotide formulations may include disposing the cannula in a dispense position, and introducing the high concentration defibrotide formulation under pressure to expand the bladder such that the second end of the bladder moves along an arcuate path around the central portion of the housing and an elastic force in the wall of the bladder expels the high concentration defibrotide formulation from the bladder and through the cannula at a flow rate between about 0.5 ml/hour to about 700 ml/hour, or between about 10 ml/hour to about 600 ml/hour, about 10 ml/hour to about 550 ml/hour, about 10 ml/hour to about 500 ml/hour, about 10 ml/hour to about 400 ml/hour, about 10 ml/hour to about 300 ml/hour, about 10 ml/hour to about 200 ml/hour, about 10 ml/hour to about 150 ml/hour, about 10 ml/hour to about 100 ml/hour, about 10 ml/hour to about 80 ml/hour, about 10 ml/hour to about 40 ml/hour, about 15 ml/hour to about 35 ml/hour, or about 15 ml/hour to about 150 ml/hour. In some embodiments, the flow rate may be about 0.5 ml/hour, about 1 ml/hour, about 5 ml/hour, about 10 ml/hour, about 15 ml/hour, about 18 ml/hour, about 20 ml/hour, about 30 ml/hour, about 40 ml/hour, about 50 ml/hour, about 60 ml/hour, about 70 ml/hour, about 80 ml/hour, about 90 ml/hour, about 100 ml/hour, about 110 ml/hour, about 120 ml/hour, about 130 ml/hour, about 140 ml/hour, about 145 ml/hour, about 148 ml/hour, about 150 ml/hour, about 160 ml/hour, about 170 ml/hour, about 180 ml/hour, about 190 ml/hour, about 200 ml/hour, about 250 ml/hour, about 300 ml/hour, about 350 ml/hour, about 400 ml/hour, about 450 ml/hour, about 500 ml/hour, about 550 ml/hour, about 600 ml/hour, or about 700 ml/hour. In one embodiment, it may be useful for the cannula flow rate to be about 20 ml/hour±10 ml/hour. In another embodiment, it may be useful for the cannula flow rate to be about 148 ml/hour. In some embodiments, the cannula inner diameter may be about 0.08 millimeters (“mm”), 0.10 mm, about 0.12 mm, about 0.13 mm, about 0.14 mm, about 0.15 mm, about 0.16 mm, about 0.17 mm, about 0.18 mm, about 0.19 mm, about 0.20 mm, about 0.21 mm, about 0.22 mm, about 0.23 mm, about 0.24 mm, about 0.25 mm, about 0.26 mm, about 0.27 mm, about 0.29 mm, about 0.30 mm, about 0.31 mm, about 0.35 mm, about 0.40, about 0.41 mm, or 0.5 mm. In one embodiment, it may be useful for the cannula inner diameter to be about 0.17 mm. In another embodiment, it may be useful for the cannula inner diameter to be about 0.27 mm. In one embodiment, the cannula inner diameter is from about 0.08 mm to about 0.41 mm. In another embodiment, the cannula inner diameter is from about 0.10 mm to about 0.30 mm. In some embodiments, the cannula inner diameter may be from about 0.003 inches to about 0.016 inches. When converting millimeters to inches the conversion of 1 inch is equal to 25.4 millimeters is used.

The injection device employed in the methods described herein may further comprise a cannula actuator coupled to the cannula of the injection device for moving the cannula, where the high concentration defibrotide formulation is administered by movement of the cannula from the pre-dispense position to i) the first depth position; and ii) to the second depth position; and then 3) retention of the injection cannula at the second depth position. The injection devices may also include a manifold disposed within the housing and coupled to the first end of the bladder, the manifold including a fluid inlet port configured to receive the high concentration defibrotide formulation and a fluid pathway configured for fluid communication between the fluid inlet port and the bladder for introduction of the high concentration defibrotide formulation under pressure into the bladder. Additionally, the injection devices may include an indicator disposed on the second end of the bladder that provides an indication of defibrotide dosing progress to the patient and/or a mandrel coupled to the second end of the bladder that pre-stresses the bladder to an expanded condition. An adhesive may be disposed on the injection device housing to help attach the injection device to the patient for the duration of treatment.

In one embodiment, the method of administering includes disposing the cannula in the dispense position and introducing the high concentration defibrotide formulation under pressure to expand the bladder such that movement of the second end of the bladder along an arcuate path results in constant-pressure driven dose delivery.

In some embodiments, the high concentration defibrotide formulation further comprises a buffer or excipient selected from sodium citrate, sodium succinate, histidine (“HIS”), TRIS buffer, HEPES buffer, sodium chloride, succinic acid, acetic acid, phosphoric acid, tartaric acid, arginine, lidocaine, benzyl alcohol, salts (e.g calcium chloride or magnesium chloride; sodium or phosphate salts),amino acids, cyclodextrin and derivatives, Captsiol®, Polyvinylpyrrolidone (PVP), Kolloidon 12 PF, Kolloidon 17PF (BASF), Kolliphor HS 15 (BASF), MES buffer, Macrogol (15) hydroxystearate, polyethylene glycol (15)-hydroxystearate, polyoxyethylated 12-hydroxystearic acid, Solutol HS 15, and/or polysorbate-80. In some embodiments, the high concentration defibrotide formulation comprises a buffer or excipient so that the nucleic acid is in the form of an alkali metal salt. In some embodiments, the buffer or excipient is sodium citrate, sodium succinate, or sodium chloride. In some embodiments, the buffer or excipient is sodium citrate, sodium succinate, or sodium chloride at a concentration of less than about 80 mM sodium salt. In some embodiments, the buffer or excipient is sodium citrate at a concentration of between about 20 mM and about 34 mM. In certain embodiments, the buffer or excipient is a buffering system from pH 6 to pH 8. In some embodiments, the buffer or excipient has a pKa value of from between 5 to 10. In some embodiments, the buffer or excipient is citrate and has pKa values of 3.13, 4.76 and 6.4. In some embodiments, the buffer or excipient is succinic acid and has pKa values of 4.22 and 5.64. In some embodiments, the buffer or excipient is MES buffer and has a pKa value of 6.15. In some embodiments, the buffer or excipient is acetic acid and has a pKa value of 4.76. In some embodiments, the buffer or excipient is phosphoric acid and has a pKa value of 7.22. In some embodiments, the buffer or excipient is tartaric acid and has pKa values of 3.04 and 4.37. In some embodiments, the buffer or excipient is Tris buffer and has a pKa value of 8.072. In some embodiments, the buffer or excipient is HEPES buffer and has a pKa value of 7.564. In some embodiments, the buffer or excipient is lidocaine and has a pKa value of 7.7. In some embodiments, the buffer or excipient is benzyl alcohol and has a pKa value of 15.4. In some embodiments, the buffer or excipient is 0.1% to 2% benzyl alcohol. In some embodiments, the buffer or excipient is an amino acid at a concentration from 5 mM to 200 mM. In some embodiments, the buffer or excipient is an amino acid selected from those listed in Table 1 below (the pKa values and the isoelectronic point, pI, are given for the 20 α-amino acids; pKa1=α-carboxyl group, pKa2=α-ammonium ion, and pKa3=side chain group):

TABLE 1

| Amino acid | pKa₁ | pKa₂ | pKa₃ | pI |
|---|---|---|---|---|
| Glycine | 2.34 | 9.60 | — | 5.97 |
| Alanine | 2.34 | 9.69 | — | 6.00 |
| Valine | 2.32 | 9.62 | — | 5.96 |
| Leucine | 2.36 | 9.60 | — | 5.98 |
| Isoleucine | 2.36 | 9.60 | — | 6.02 |
| Methionine | 2.28 | 9.21 | — | 5.74 |
| Proline | 1.99 | 10.6 | — | 6.30 |
| Phenylalanine | 1.83 | 9.13 | — | 5.48 |
| Tryptophan | 2.83 | 9.39 | — | 5.89 |
| Asparagine | 2.02 | 8.80 | — | 5.41 |
| Glutamine | 2.17 | 9.13 | — | 5.65 |
| Serine | 2.21 | 9.15 | — | 5.68 |
| Threonine | 2.09 | 9.10 | — | 5.60 |
| Tyrosine | 2.2 | 9.11 | — | 5.66 |
| Cysteine | 1.96 | 8.18 | — | 5.07 |
| Aspartic acid | 1.88 | 9.60 | 3.65 | 2.77 |
| Glutamic acid | 2.19 | 9.67 | 4.25 | 3.22 |
| Lysine | 2.18 | 8.95 | 10.53 | 9.74 |
| Arginine | 2.17 | 9.04 | 12.48 | 10.76 |
| Histidine | 1.82 | 9.17 | 6.00 | 7.59 |

In some embodiments, the viscosity of the high concentration defibrotide formulation decreases over time. In some of the embodiments, the viscosity of the high concentration defibrotide formulation decreases during storage. In certain embodiments, the viscosity of the high concentration defibrotide formulation decreases during storage up to about 85% of its initial (time=T0) viscosity under room temperature storage conditions. In some embodiments, the viscosity of the high concentration defibrotide formulation decreases under the increasing rate of shear or increases in agitation, and/or pressure. In some embodiments, said shear increases during administration. In some embodiments, the rate of shear increases during administration via a needle or device. In some embodiments, the rate of shear increases during administration due to the pressure variations during treatment.

In some embodiments, the high concentration defibrotide formulation is formulated for subcutaneous delivery.

In some embodiments, the high concentration defibrotide formulation given subcutaneously demonstrates extended systemic half-life compared to an intravenously administered defibrotide formulation. In some embodiments, the high concentration defibrotide formulation given subcutaneously exhibits lower peak-to-trough ratios of plasma concentrations compared to an intravenously administered defibrotide formulation. In some embodiments, the high concentration defibrotide formulation given subcutaneously exhibits improved Cmax and/or AUC compared to an intravenously administered defibrotide formulation. In some embodiments, the high concentration defibrotide formulation given subcutaneously exhibits improved efficacy and/or an improved safety profile compared to an intravenously administered defibrotide formulation.

In some embodiments, the high concentration defibrotide formulation is isotonic, hypertonic or thixotropic.

In some embodiments, the high concentration defibrotide formulation comprises about 120-200 mg/mL of defibrotide and about 10-100 mM viscosity reducer, and is formulated for subcutaneous delivery to a patient. In some embodiments, the high concentration defibrotide formulation further comprises between about 10 mM to about 34 mM sodium citrate. In some embodiments, the high concentration defibrotide formulation comprises about 180 mg/ml of defibrotide, about 10-100 mM viscosity reducer, and about 10-25 mM sodium citrate. In some embodiments, the high concentration defibrotide formulation comprises about 120-200 mg/mL of defibrotide, about 10-100 mM viscosity reducer, and about 10-25 mM sodium citrate, wherein the formulation is formulated for subcutaneous or intravenous delivery to a patient. Preferably, the viscosity reducer is glycylglycine, glycine, or sodium citrate.

In some embodiments, an agent which improves processing, such as a wetting agent, is included in high concentration defibrotide formulations of the invention. In specific embodiments, hyaluronidase is co-administered or co-formulated with a high concentration defibrotide formulation of the invention.

In some embodiments, the high concentration defibrotide formulation comprises about 120-200 mg/mL of defibrotide, about 10-100 mM viscosity reducer, and about 25-35 mM sodium citrate, wherein the formulation is formulated for subcutaneous or intravenous delivery to a patient. In some embodiments, the viscosity reducer is glycylglycine, glycine, sodium citrate, benzyl alcohol or a hyaluronidase (e.g. PH20).

In some aspects of the present disclosure, the high concentration defibrotide formulation is administered to treat or prevent a disease or condition selected from thrombosis, Hematopoietic Stem Cell Transplantation (HSCT) related complications including sinusoidal obstruction syndrome or hepatic veno-occlusive disease (VOD), Graft versus Host Disease (GvHD), Transplant-Associated Thrombotic Microangiopathy (TA-TMA) or Idiopathic Pneumonia Syndrome, other TMAs including Thrombotic Thrombocytopenic Purpura (TTP) and Hemolytic-Uremic Syndrome (HUS), Acute Myocardial Ischemia, Ischemic Stroke (including acute ischemic stroke), Ischemia Reperfusion Injury (IRI, including kidney IRI), cytokine release syndrome (CRS) or Chimeric Antigen Receptor (CAR)-T Cell Related Encephalopathy Syndrome (CRES) or CAR-T neurotoxicity, Acute Respiratory Distress Syndrome (ARDS), Sickle Cell Vaso-occlusive Crisis (VOC), Sickle Cell Related Acute Chest Syndrome, Disseminated Intravascular Coagulation (DIC), Sepsis, Renal Insufficiency, other Coronary or Peripheral Artery Diseases, Hematological Malignancies or Solid Tumors.

In some embodiments, the high concentration defibrotide formulation is administered at a dosing regimen that provides improved patient quality of life by including a reduced administration volume and/or improved convenience of administration on an outpatient basis, e.g., by allowing less-frequent administration and/or a shorter duration of administration.

In some aspects, the present disclosure provides automated injection devices for delivery of a high concentration defibrotide formulation, wherein the automated injection device generally includes: a) a housing defining a central portion, wherein the housing has a circular profile and includes a spiral recess;

b) a bladder positioned within the housing, the bladder including a first end, a second end, and a wall extending between the first end and the second end, the bladder being expandable, where the bladder wall includes an area of increased thickness along a side of the bladder wall, where the area of increased thickness deflects less than a remaining portion of the bladder wall during administration to effect the movement of the distal end of the bladder along an arcuate path; and c) a cannula disposed within the central portion of the housing and configured to be movable between multiple positions, including a pre-dispense position where the cannula is not in fluid communication with the bladder, and a dispense position where the cannula is in fluid flow communication with the bladder. In some instances, the cannula includes a side hole configured so that when the cannula is in the pre-dispense position or in a first depth position with respect to the surface of a patient's skin, the side hole is not in fluid communication with the bladder, and when the injection cannula is in a second depth position with respect to the surface of a patient's skin, the side hole is in fluid flow communication with the bladder. The injection devices described herein are generally combination products for the delivery or administration of high concentration defibrotide formulations also described herein.

In some embodiments, the cannula may be coated with an agent to prevent potential occlusions. In specific embodiments, the cannula may be coated with heparin.

The cannula may have an inner diameter that ranges from about 0.08 mm to about 0.41 mm, or from about 0.1 mm to about 0.3 mm. The automated injection devices may further comprise a septum positioned within the housing and configured to seal around a periphery of the side hole of the cannula when the cannula is in the pre-dispense position or in the first depth position.

In some embodiments, the automated injection devices further include a cannula actuator coupled to the cannula for moving the cannula, where the high concentration defibrotide formulation is administered by movement of the cannula from the pre-dispense position to i) the first depth position; and ii) to the second depth position; and then 3) retention of the injection cannula at the second depth position.

In other embodiments, the automated injection devices may further comprise a manifold disposed within the housing and coupled to the first end of the bladder, the manifold including a fluid inlet port configured to receive the high concentration defibrotide formulation and a fluid pathway configured for fluid communication between the fluid inlet port and the bladder for introduction of the high concentration defibrotide formulation under pressure into the bladder.

Other features that may be included with the automated injection devices include an indicator disposed on the second end of the bladder that provides an indication of dosing progress to the patient, a mandrel coupled to the second end of the bladder for pre-stressing the bladder to an expanded condition, and an adhesive disposed on the injection device housing for attaching the injection device to the patient.

Methods of preparing a high concentration defibrotide formulation are also disclosed using the automated injection devices described herein.

The injections devices are a combination product for the subcutaneous delivery of a high concentration defibrotide formulation, as previously mentioned. The high concentration defibrotide formulations may comprise between 100 mg/mL to about 400 mg/mL of defibrotide, and a viscosity reducer at a concentration of between about 5 mM and about 100 mM, where the formulation has a viscosity between about 5 and about 70 cP when measured at a temperature between 15° C. and 25° C., and an osmolality between about 240 mOsm/kg and about 1000 mOsm/kg. In some embodiments, the viscosity reducer is glycylglycine, glycine, sodium citrate or benzyl alcohol.

The cannula of the injection devices may have an inner diameter between 0.10 mm and about 0.30 mm, or about 0.15 mm to about 0.30 mm, resulting in a flow rate between about 0.5 ml/hour to about 700 ml/hour, or between about 15 ml/hour to about 150 ml/hour.

In some embodiments, the cannula inner diameter may be about 0.08 mm, 0.10 mm, about 0.12 mm, about 0.13 mm, about 0.14 mm, about 0.15 mm, about 0.16 mm, about 0.17 mm, about 0.18 mm, about 0.19 mm, about 0.20 mm, about 0.21 mm, about 0.22 mm, about 0.23 mm, about 0.24 mm, about 0.25 mm, about 0.26 mm, about 0.27 mm, about 0.29 mm, about 0.30 mm, about 0.31 mm, about 0.35 mm, about 0.40, about 0.41 mm, or 0.5 mm. In one embodiment, it may be useful for the cannula inner diameter to be about 0.17 mm. In another embodiment, it may be useful for the cannula inner diameter to be about 0.27 mm.

With respect to flow rate, the bladder of the injection device may expel the high concentration defibrotide formulation through the cannula at a flow rate between about 0.5 ml/hour to about 700 ml/hour, or between about 15 ml/hour to about 150 ml/hour. In some embodiments, the flow rate may be about 0.5 ml/hour, about 1 ml/hour, about 5 ml/hour, about 10 ml/hour, about 15 ml/hour, about 18 ml/hour, about 20 ml/hour, about 30 ml/hour, about 40 ml/hour, about 50 ml/hour, about 60 ml/hour, about 70 ml/hour, about 80 ml/hour, about 90 ml/hour, about 100 ml/hour, about 110 ml/hour, about 120 ml/hour, about 130 ml/hour, about 140 ml/hour, about 145 ml/hour, about 148 ml/hour, about 150 ml/hour, about 160 ml/hour, about 170 ml/hour, about 180 ml/hour, about 190 ml/hour, about 200 ml/hour, about 250 ml/hour, about 300 ml/hour, about 350 ml/hour, about 400 ml/hour, about 450 ml/hour, about 500 ml/hour, about 550 ml/hour, about 600 ml/hour, or about 700 ml/hour. In one embodiment, it may be useful for the cannula flow rate to be about 15 ml/hour to about 35 ml/hour. In one embodiment, it may be useful for the cannula flow rate to be about 20±10 ml/hour. In another embodiment, it may be useful for the cannula flow rate to be about 148 ml/hour.

The methods and delivery device in combination with high concentration, low viscosity formulations of the instant disclosure offer numerous benefits to the patient, including for example, the ability to subcutaneously administer the formulations and administration of the formulations to the patient outside of a hospital setting. The advantages of self-administration and subcutaneous administration are felt by the patient and their families as well as by the hospital. The amount of time and resources that the hospital needs to treat and monitor these patients are significantly reduced which provides a reduced economic burden on both the hospital and the patient.

Provided herein are high concentration low-viscosity defibrotide formulations for therapeutic subcutaneous administration via an automated injection device, and which may improve the quality of life for patients by less frequent and/or shorter duration of dosing than similar defibrotide products currently on the market.

### BRIEF DESCRIPTION OF THE DRAWINGS

FIG. 1 is a perspective view of an exemplary injection device.

FIGS. 2A and 2B show the exemplary injection device of FIG. 1 with the upper housing removed and the expandable member in a filled state. FIG. 2A is a perspective view and FIG. 2B is a top view of the injection device.

FIG. 3 shows the exemplary injection device of FIG. 1 with the upper housing removed and the expandable member in an empty state.

FIGS. 4A-4C depict cross-sectional views of an exemplary injection device with the cannula in a pre-fire state (FIG. 4A), a first depth position (FIG. 4B), and a second depth position (FIG. 4C).

### DETAILED DESCRIPTION

Defibrotide (CAS number 83712-60-1) is a substance derived from materials of natural origin. It is the sodium salt of relatively low molecular weight polydeoxyribonucleotides which are obtained by extraction from animal mucosa. Defibrotide has a diverse size range and is known to have a mean molecular weight (MW) between 13 and 20 kDa. Defibrotide can be obtained according to U.S. Pat. Nos. 4,985,552 and 5,223,609 and/or presents the physical/chemical characteristics described in the same U.S. Pat. Nos. 4,985,552 and 5,223,609, each of which is incorporated herein by reference. Synthetic defibrotide, presented as phosphodiester oligonucleotides that mimic the therapeutic action of defibrotide are described in US20110092576 which is incorporated herein by reference in its entirety.

Defibrotide has numerous therapeutic applications, including use as an anti-thrombotic agent (U.S. Pat. No. 3,829,567), treatment of peripheral arteriopathies, treatment of acute renal insufficiency (U.S. Pat. No. 4,694,134), and treatment of acute myocardial ischaemia (U.S. Pat. No. 4,693,995). More recently, defibrotide has been used for the treatment and prevention of sinusoidal obstruction syndrome/veno occlusive disease (EU clinical trial EudraCT:2004-000592-33, US clinical trial 2005-01 (ClinicalTrials.gov identifier: NCT00358501). Patients are treated with a 6.25 mg/kg dose given as a two hour intravenous infusion every six hours until signs and symptoms of VOD are mitigated. As mentioned above, Defibrotide is currently sold under the name Defitelio® as a single vial for injection (commercially available from Gentium S.r.L., Villa Guardia, Italy; see package insert available at dailymed.nlm.nih.gov/dailymed/search.cfm?labeltype=all&query=defibrotide). Defitelio® is prepared as an intravenous infusion by a dilution in 5% Dextrose Injection, USP or 0.9% Sodium Chloride Injection, USP. Intravenous preparation is used within 4 hours if stored at room temperature or within 24 hours if stored under refrigeration. It is administered for a total of 8 hours over 4 intravenous infusions.

The development of novel defibrotide high concentration low viscosity formulations for administration via an automated injection device (point of use filled, prefilled) using syringes or glass or plastic ampules with or without silicone oil) offer improved quality of life for the patients undergoing treatment. For example, decreasing the frequency from 4 times daily to once or twice daily as well as decreasing the duration of the infusions may offer quality of life improvements to patients while being treated. SC route of administration of a high concentration low viscosity defibrotide formulation via an automated injection device may offer significant reduction of the time for clinical administration and enable outpatient dosing of the product for as long as needed. The use of an automated injection device improves convenience and allows faster administration by health-care professionals (HCP), care-givers, or even self-administration by the patients.

In some embodiments, the route of administration affects the efficacy and/or longevity of the formulations of the present disclosure. In some embodiments, subcutaneous administration is associated with an extended systemic half-life compared to the same formulation administered intravenously. In some embodiments, subcutaneous administration of the formulation provides lower peak-to-trough ratios of plasma concentrations compared to the same formulation administered intravenously. In some embodiments, subcutaneous administration provides improved efficacy and/or improves the safety profile of the formulation compared to the same formulation administrated intravenously.

### Definitions

The following definitions are given for a better understanding of the present disclosure:

As used herein, the term “defibrotide” refers to both natural and synthetic sources of defibrotide, including synthetic phosphodiester oligonucleotides as described in US patent application number 20110092576. The term defibrotide identifies a polydeoxyribonucleotide that is obtained by extraction from animal and/or vegetable tissues but which may also be produced synthetically; the polydeoxyribonucleotide is normally used in the form of an alkali-metal salt, generally a sodium salt, and generally has a molecular weight of 13 to 30 kDa (CAS Registry Number: 83712-60-1). Preferably, defibrotide is obtained according to U.S. Pat. Nos. 4,985,552 and 5,223,609 and/or presents the physical/chemical characteristics described in the same U.S. Pat. Nos. 4,985,552 and 5,223,609, herein incorporated by reference. More in particular, defibrotide is a mixture of polydeoxyribonucleotides having formula of random sequence: P1-5, (dAP)₁₂₋₂₄, (dGP)₁₀₋₂₀, (dPp)₁₃₋₂₆, (dCP)₁₀₋₂₀, where: P=phosphoric radical; dAp=deoxyadenylic monomer; dGp=deoxyguanylic monomer; dTp=deoxythymidinic monomer; dCp=deoxycytidynic monomer; and/or shows the following chemical/physical characteristics: electrophoresis=homogeneous anodic mobility, and/or extinction coefficient, E₁ cm^{1%} at 260±1 nm nm=220±10, and/or E₂₃₀/E₂₆₀=0.45±0.04, and/or coefficient of molar extinction (referred to phosphorous) ε(P)=7.750±500, and/or rotatory power [α]_{D}²⁰°=53°±6; and/or reversible hyperchromicity, indicated as % in native DNA and/or h=15±5.

As used herein, the term “polydeoxyribonucleotide” refers to a polymer whose constituent monomer is a deoxyribonucleotide.

As used herein, the term “oligodeoxyribonucleotide” refers to any oligonucleotide composed of deoxyribose monomers.

As used herein, the term “mean MW” refers to the mean or average molecular weight of the polymer.

The term, “glycylglycine” or “Gly-Gly” or “GlyGly” or “glygly” as used herein, refers to a simple peptide, made of two glycine molecules (glycine is a simple, nonessential amino acid); the dipeptide is used in the synthesis of more complicated peptides. Glycylglycine, an ampholyte, is also sometimes referred to as Diglycine, Diglycocoll, Glycine dipeptide, N-Glycylglycine. It can be made by methods such as those described in CN patent application 101759767 which is incorporated herein by reference in its entirety.

The term, “excipient,” as used herein, refers to any substance that may be formulated with defibrotide and may be included for the purpose of enhancement of the defibrotide in the final dosage form, such as facilitating its bioavailability, reducing viscosity and/or osmolality, enhancing solubility of the composition or to enhance long-term stability. Excipients can also be useful in the manufacturing process, to aid in the handling of the active substance. The selection of appropriate excipients also depends upon the route of administration and the dosage form, as well as the active ingredient and other factors. Accordingly, defibrotide may be combined with any excipient(s) known in the art that allows tailoring its performance during manufacturing or administration as well as its in vitro and in vivo performance. Many of these excipients may be utilized to tailor the pharmacokinetic profiles of defibrotide formulations.

The term, “buffer” or “buffering agent,” as used herein, refers to a solution which resists changes in the hydrogen ion concentration on the addition of a small amount of acid or base. This includes, for example, a weak acid or base that is used to maintain the pH of a solution near a chosen pH value after the addition of another acidic or basic compound. The function of such buffer or buffering agent is to prevent a change in pH of a solution when acids or bases are added to said solution.

The term, “pH adjusting agent,” as used herein, refers to an acid or base used to alter the pH of a solution to a chosen pH value. The function of such an agent is to alter the pH of a solution to the desired value subsequent to the addition of acidic or basic compounds.

The term, “formulation,” as used herein, refers to compositions for therapeutic use, including, for example, a stable and pharmaceutically acceptable preparation of a pharmaceutical composition or formulation disclosed herein.

The term, “low-viscosity formulation,” as used herein, refers to a formulation which has a viscosity that is less than about 100 centipoise (cP). Normally viscosity is measured at ambient/room temperatures of (e.g. 15° C. to 35° C.; between 18° C. to 25° C. or between 21° C. to 23° C.) depending on the geographic region and/or weather conditions of the room in which it is being measured.

The term, “viscosity reducer,” as used herein, refers to a buffer, excipient or other agent which acts to lower or stabilize viscosity, for example, glycylglycine, glycine, sodium citrate, sodium succinate, histidine, TRIS buffer, HEPES buffer, sodium chloride, arginine, lidocaine, benzyl alcohol, polysorbate-80, and/or agents such as a hyaluronidase, including for example PH20, which can help stabilize the formulation. For example, the presence of a viscosity reducer as used herein delays or prevents viscosity increases at ambient/room temperatures and/or 4° C.

The term, “aqueous formulation,” as used herein, refers to a water-based formulation, in particular, a formulation that is an aqueous solution.

The term, “high concentration formulation” or “high concentration liquid formulation” “HCLF” or “high concentration low-viscosity formulation” as used herein, refers to those formulations where the concentration of the nucleic acid is about 80 mg/mL or higher; or about 85 mg/mL or higher and includes both aqueous and non-aqueous formulations.

The term, “high concentration defibrotide formulations” or “HCLF of defibrotide” as used herein, refers to those formulations where the defibrotide concentration is about 80 mg/mL or higher; or about 85 mg/mL or higher.

The term “combination-product” or “combination-product of the high concentration liquid defibrotide formulation and the injection device” refers to a combination of high concentration defibrotide with an injection device as described herein and which is claimed in the present invention. The term “Defibrotide and Wearable Infusion Combination Product” may also be used for such purposes.

The term, “pharmacokinetic” or “PK” as used herein, refers to in vivo movement of an individual agent in the body, including the plasma concentration time profiles and kinetic parameters like the maximum concentration (Cmax), area under the curve (AUC), and time to maximum concentration of said agent (Tmax).

The phrase “pharmaceutically acceptable” or “acceptable”, as used in connection with compositions of the disclosure, refers to molecular entities and other ingredients of such compositions that are physiologically tolerable and do not typically produce untoward reactions when administered to an animal and/or human. Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The term “physiologically relevant” as used herein, refers to a measurement, level or amount that is suitable for use in a pharmaceutical, therapeutic or other dosage form to be administered to an animal subject, particularly a human subject.

As used herein, the term “parenteral” refers to any non-oral means of administration. It includes intravenous (i.v. or IV) infusion, IV bolus injection, subcutaneous (s.c. or SC) and intramuscular (i.m. or IM) injection.

As used herein, the terms “administering” or “administration” are intended to encompass all means for directly and indirectly delivering a compound to its intended site of action.

As used herein, the term “animal” means any animal, including mammals and, in particular, humans.

As used herein, the term “patient” refers to a mammal, particularly a human. Patients to be treated by the methods of the disclosed embodiments include both human subjects and animal subjects (e.g., dog, cat, monkey, chimpanzee, and/or the like) for veterinary purposes. The patients may be male or female and may be any suitable age, e.g., neonatal, infant, juvenile, adolescent, adult, or geriatric.

The terms “treat,” “treating” or “treatment,” and the like as used herein, refers to a method of alleviating or abrogating a disease and/or its attendant symptoms. For example, within the meaning of the present disclosure, the term “treat” also denotes to arrest, delay the onset (i.e., the period prior to clinical manifestation of a disease) and/or reduce the risk of developing or worsening a disease.

The terms “a” and “an,” when used to modify the ingredient of a composition, such as, active agent, buffering agent, and osmolyte, do not denote a limitation of quantity, but rather denote the presence of at least one of the referenced item. The term “or” or “and/or” is used as a function word to indicate that two words or expressions are to be taken together or individually. The terms “comprising,” “having,” “including,” and “containing” are to be construed as open-ended terms (i.e., meaning “including, but not limited to”). The endpoints of all ranges directed to the same component or property are inclusive and independently combinable.

Throughout the present specification, the terms “about” and/or “approximately” may be used in conjunction with numerical values and/or ranges. The term “about” is understood to mean those values near to a recited value. For example, “about 1200 [units]” may mean within ±10% of 1200, within ±10%, ±9%, ±8%, ±7%, ±7%, ±5%, ±4%, ±3%, ±2%, ±1%, less than ±1%, or any other value or range of values therein. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 70-70, etc.). Furthermore, all values within a given range may be an endpoint for the range encompassed thereby (e.g., the range 50-80 includes the ranges with endpoints such as 55-80, 50-75, etc.).

High Concentration Low Viscosity Formulations

One embodiment of the present disclosure is the development of high concentration, low-viscosity liquid formulations (HCLFs) of nucleic acids and their salts for convenient drug delivery to a patient. In particular, HCLF formulations suitable for use in the invention include those described in PCT/US2018/045152 which is hereby incorporated by reference in its entirety. In addition, nucleic acid compositions which may be administered subcutaneously and/or which may require less frequent dosing than nucleic acid products currently on the market are investigated. In certain embodiments, high concentration nucleic acid formulations are self-administered on an out-patient basis (e.g. using an automated injection device of the instant disclosure). Some formulations of the disclosure have thixotropic and sheer thinning behaviors which are particularly preferred for subcutaneous administration. Formulations as provided herein offer improved tolerability, patient convenience during treatment and opportunity for outpatient dosing in comparison to currently available commercial nucleic acid formulations. In some embodiments, the viscosity of high concentration nucleic acid formulations provided herein decreases over time. In certain embodiments, the viscosity and/or fluidity of high concentration nucleic acid formulations provided herein decreases under an increase in shear strain. In certain embodiments, the viscosity and/or fluidity of high concentration nucleic acid formulations decrease with increases in temperature. It should be understood that such properties are preferable for injectables and delivery devices, such as a syringe or preloaded subcutaneous device, in which the strain or shear stress the formulation is exposed to increases as the formulation passes from the barrel of the syringe/device through to the reduced orifice of the needle. In certain embodiments, the nucleic acid is defibrotide.

Formulations of the disclosure, particularly those comprising defibrotide, may be used for the treatment of numerous conditions including, for example, treatment of peripheral arteriopathies, treatment of acute renal insufficiency, treatment of acute myocardial ischemia, treatment and prevention of Graft versus Host Disease (GvHD), treatment and prevention of Transplant-Associated Thrombotic Microangiopathy (TA-TMA), treatment of Ischemia Reperfusion Injury, such as for example, in solid organ transplantation (Kidney IRI for example), treatment and prevention of cytokine release syndrome (CRS) or Chimeric Antigen Receptor (CAR)-T Cell Related Encephalopathy Syndrome (CRES), and treatment and prevention of sinusoidal obstruction syndrome or VOD. In some embodiments, formulations of the disclosure, particularly those comprising defibrotide, may be administered to patients who have undergone, are undergoing, or are about to undergo, chemotherapy, stem cell ablation, and/or hematopoietic stem cell transplantation (HSCT). Other uses of defibrotide, methods for its production and testing are described in the following patents, patent applications and articles, each of which is hereby incorporated by reference in its entirety: U.S. Pat. Nos. 3,770,720; 3,829,567; 3,899,481; 4,693,134; 4,693,995; 4,938,873; 4,985,552; 5,081,109; 5,116,617; 5,223,609; 5,646,127; 5,646,268; 5,977,083; 6,046,172; 6,699,985; 6,767,554; 7,338,777; 8,551,967; 8,771,663, US Patent Publication Nos. 20080194506; 20090131362; 20110092576; 20130231470; 20140005256, U.S. patent application Ser. Nos. 14/019,674; 14/323,918; 14/408,272; 16/105,319; 62/656,486; 62/657,161; 62/664,657; and International applications WO 2013/190582, WO 2019/028340, and PCT/EP2015/077355. See also Palmer and Boa, Defibrotide. A Review of its pharmacodynamic and pharmacokinetic properties, and therapeutic use in vascular disorders, Drugs, 1993, February; 45(2):259-94; which is incorporated by reference herein. Other references cited throughout are also incorporated by reference in their entireties.

In certain embodiments, the defibrotide to be evaluated by the methods described herein are manufactured by a process such as that described in U.S. Pat. Nos. 4,985,552 and 5,223,609, both of which are hereby incorporated by reference in their entireties. In one preferred embodiment of the disclosure n, defibrotide is a polydeoxyribonucleotide corresponding to the following formula of random sequence:

P1-5, (dAp)12-24,(dGp)10-20,(dTp)13-26,(dCp)10-20

wherein: P=phosphoric radical

dAp=deoxyadenylic monomer

dGp=deoxyguanylic monomer

dTp=deoxythymidylic monomer

dCp=deoxycytidylic monomer.

The defibrotide as used herein may have one or more or all of the following chemico-physical properties: electrophoresis=homogeneous anodic mobility; extinction coefficient, E1 cm1% at 260±1 nm=220±10; extinction ratio, E230/E260=0.45±0.04; coefficient of molar extinction (referred to phosphorus), ε(P)=7.750±500; rotary power [α]D20°=53°±6; reversible hyperchromicity, indicated as % in native DNA, h=15±5; and a purine:pyrimidine ratio of 0.95±0.5.

In some aspects, the present disclosure provides a nucleic acid formulation with various buffers or excipients, such as those found in Remington, The Science and Practice of Pharmacy (Remington the Science and Practice of Pharmacy) Twenty-Second Edition, 2013 Pharmaceutical Press which is hereby incorporated by reference in its entirety. See especially the monograph on Excipients starting at page 1837. Preferably, the nucleic acid is defibrotide. In some embodiments, a nucleic acid other than defibrotide is used. In some embodiments, the present disclosure provides a high concentration low viscosity formulation comprising a nucleic acid. In some embodiments, the present disclosure provides a high concentration low viscosity formulation comprising defibrotide.

In some embodiments, the disclosure includes a dipeptide buffer (e.g. L-Carnosine or glycylglycine). In some embodiments of the disclosure the dipeptide buffer includes glycylglycine, which is a dipeptide of glycine. It is commercially available from supply houses, such as Sigma-Aldrich, and is useful as an excipient for biological systems. In some embodiments of the present disclosure, glycylglycine is present at concentrations between about 1 mM to about 50 mM. In some embodiments, glycylglycine is present at concentrations between about 5 mM to about 100 mM, about 10 to about 60 mM, or about 10 to about 40 mM. In some embodiments, the glycylglycine is present at a concentration of about 1 mM, about 5 mM, about 10 mM, about 15 mM, about 20 mM, about 25 mM, about 30 mM, about 35 mM, about 40 mM, about 45 mM, or about 50 mM.

In other embodiments, the high concentration nucleotide formulation comprises glycine at concentrations between about 5 mM to about 100 mM, about 10 to about 60 mM, or about 10 to about 40 mM.

In further embodiments, the high concentration nucleotide formulation comprises benzyl alcohol at concentrations between about 5 mM to about 100 mM, about 10 to about 60 mM, or about 10 to about 40 mM.

In some embodiments, the high concentration nucleotide formulation comprises a viscosity reducer at concentrations between about 5 mM and about 100 mM. In some embodiments, the formulation comprises a viscosity reducer at a concentration of about 5 mM, about 6 mM, about 7 mM, about 8 mM, about 9 mM, 10 mM, about 11 mM, about 12, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM, about 79 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, or about 100 mM. In some embodiments, the viscosity reducer is glycylglycine, glycine, sodium citrate or benzyl alcohol.

Other buffers or excipients can be present in the present formulation. In some embodiments, the one or more excipients is a viscosity-reducer In some embodiments, the high concentration defibrotide formulation further comprises a buffer or excipient selected from sodium citrate, sodium succinate, histidine (“HIS”), TRIS buffer, HEPES buffer, sodium chloride, succinic acid, acetic acid, phosphoric acid, tartaric acid, arginine, lidocaine, benzyl alcohol, salts (e.g calcium chloride or magnesium chloride; sodium or phosphate salts), amino acids, cyclodextrin and derivatives, Captsiol®, Polyvinylpyrrolidone (PVP), Kolloidon 12 PF, Kolloidon 17PF (BASF), Kolliphor HS 15 (BASF), MES buffer, Macrogol (15) hydroxystearate, polyethylene glycol (15)-hydroxystearate, polyoxyethylated 12-hydroxystearic acid, Solutol HS 15, Benzyl alcohol, and/or polysorbate-80. In some embodiments, the low-viscosity formulation comprises a buffer or excipient so that the nucleic acid is in the form of an alkali metal salt. In some embodiments, the buffer or excipient includes a sodium salt. In some embodiments, the buffer or excipient is sodium citrate, sodium succinate, or sodium chloride.

In some embodiments, the buffer or excipient is sodium citrate, sodium succinate, or sodium chloride at a concentration of less than about 80 mM sodium salt. In some embodiments, the formulation comprises about 1-80 mM sodium salt. In some embodiments, the formulation comprises about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, or about 50 mM sodium salt.

In some embodiments, the formulation comprises sodium citrate. In some embodiments, the sodium citrate is present at concentrations between about 5 to about 50 mM between about 5 to about 60 mM, about 10 to about 60 mM, about 10 to about 40 mM or about 10 mM to about 34 mM. In some embodiments, the concentration of sodium citrate is about a 1 mM, about 5 mM, about 10 mM, about 15 mM, about 20 mM, about 25 mM, about 30 mM, about 35 mM, about 40 mM, about 45 mM, about 50 mM, about 55 mM, or about 60 mM. In some embodiments, the sodium citrate is present at concentrations of about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, about 55 mM, about 56 mM, about 57 mM, about 58 mM, about 59 mM or about 60 mM.

In some embodiments, the high concentration low-viscosity formulation comprises salts such as calcium chloride, magnesium chloride, counterions such as Ca²⁺, Cl⁻, Mg²⁺, hoffmeister series (e.g. F—, SO₄²⁻, HPO₄²⁻, acetate, Cl—, NO₃⁻, Mg²⁺, Li+, Na+, K+, NH₄⁺. In some embodiments, the high concentration low-viscosity formulation comprises a salt in a concentration of about 10 mM to about 200 mM or from about 10 mM to about 50 mM, or from about 50 mM to about 150 mM. In some embodiments, the high concentration low-viscosity formulation comprises a salt in a concentration of about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM, about 79 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, about 100 mM, about 101 mM, about 102 mM, about 103 mM, about 104 mM, about 105 mM, about 106 mM, about 107 mM, about 108 mM, about 109 mM, about 110 mM, about 111 mM, about 112 mM, about 113 mM, about 114 mM, about 115 mM, about 116 mM, about 117 mM, about 118 mM, about 119 mM, about 120 mM, about 121 mM, about 122 mM, about 123 mM, about 124 mM, about 125 mM, about 126 mM, about 27 mM, about 28 mM, about 129 mM, about 130 mM, about 131 mM, about 132 mM, about 133 mM, about 34 mM, about 35 mM, about 136 mM, about 137 mM, about 138 mM, about 139 mM, about 140 mM, about 41 mM, about 42 mM, about 143 mM, about 144 mM, about 145 mM, about 146 mM, about 147 mM, about 48 mM, about 48 mM, about 149 mM, about 150 mM, about 151 mM, about 152 mM, about 153 mM, about 54 mM, about 55 mM, about 156 mM, about 157 mM, about 158 mM, about 159 mM, about 160 mM, about 61 mM, about 62 mM, about 163 mM, about 164 mM, about 165 mM, about 166 mM, about 167 mM, about 168 mM, about 169 mM, about 170 mM, about 171 mM, about 172 mM, about 173 mM, about 74 mM, about 75 mM, about 76 mM, about 177 mM, about 178 mM, about 179 mM, about 180 mM, about 81 mM, about 82 mM, about 83 mM, about 184 mM, about 185 mM, about 186 mM, about 187 mM, about 88 mM, about 89 mM, about 90 mM, about 191 mM, about 192 mM, about 193 mM, about 194 mM, about 95 mM, about 96 mM, about 97 mM, about 198 mM, about 199 mM, or about 200 mM.

In some embodiments, the high concentration low-viscosity formulation comprises an amino acid. In some embodiments, the amino acid is selected from the group including, but not limited to, methionine, phenylalanine, glycine, proline, and/or serine. In some embodiments, the high concentration low-viscosity formulation comprises an amino acid at a concentration between about 5 mM to about 100 mM or about 100 mM to about 200 mM. In some embodiments, the high concentration low-viscosity formulation comprises and amino acid at a concentration of about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM, about 79 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, about 100 mM, about 101 mM, about 102 mM, about 103 mM, about 104 mM, about 105 mM, about 106 mM, about 107 mM, about 108 mM, about 109 mM, about 110 mM, about 111 mM, about 112 mM, about 113 mM, about 114 mM, about 115 mM, about 116 mM, about 117 mM, about 118 mM, about 119 mM, about 120 mM, about 121 mM, about 122 mM, about 123 mM, about 124 mM, about 125 mM, about 126 mM, about 27 mM, about 28 mM, about 129 mM, about 130 mM, about 131 mM, about 132 mM, about 133 mM, about 34 mM, about 35 mM, about 136 mM, about 137 mM, about 138 mM, about 139 mM, about 140 mM, about 41 mM, about 42 mM, about 143 mM, about 144 mM, about 145 mM, about 146 mM, about 147 mM, about 48 mM, about 48 mM, about 149 mM, about 150 mM, about 151 mM, about 152 mM, about 153 mM, about 54 mM, about 55 mM, about 156 mM, about 157 mM, about 158 mM, about 159 mM, about 160 mM, about 61 mM, about 62 mM, about 163 mM, about 164 mM, about 165 mM, about 166 mM, about 167 mM, about 168 mM, about 169 mM, about 170 mM, about 171 mM, about 172 mM, about 173 mM, about 74 mM, about 75 mM, about 76 mM, about 177 mM, about 178 mM, about 179 mM, about 180 mM, about 81 mM, about 82 mM, about 83 mM, about 184 mM, about 185 mM, about 186 mM, about 187 mM, about 88 mM, about 89 mM, about 90 mM, about 191 mM, about 192 mM, about 193 mM, about 194 mM, about 95 mM, about 96 mM, about 97 mM, about 198 mM, about 199 mM, or about 200 mM

In some embodiments, the high concentration low-viscosity formulation comprises a cyclodextrin or derivative thereof. In some embodiments, the cyclodextrin is selected from a list including, but not limited to, α (alpha)-cyclodextrin, β (beta)-cyclodextrin, γ (gamma)-cyclodextrin, and hydroxypropylbetacyclodextrin. In some embodiments, the high concentration low-viscosity formulation comprises a cyclodextrin at a concentration of about 0.5% to about 10%. In some embodiments, the high concentration formulation comprises about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, or about 10.0%.

In some embodiments, the high concentration low-viscosity formulation comprises a multitude of polymeric structures such as Captsiol® (Ligand Pharmaceuticals). In some embodiments, the high concentration low-viscosity formulation comprises Captsiol® at a concentration of about 0.5 to about 30%. In some embodiments, the high concentration low-viscosity formulation comprises Captsiol® at a concentration of about 0.5% to about 10%. In some embodiments, the high concentration formulation comprises about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, about 10.0%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%.

In some embodiments, the high concentration low-viscosity formulation comprises Polyvinylpyrrolidone (PVP), Kolloidon 12 PF, Kolloidon 17PF (BASF), and/or Kolliphor HS 15 (BASF) at a concentration of about 0.5% to about 10%. In some embodiments, the high concentration low-viscosity formulation comprises Polyvinylpyrrolidone (PVP), Kolloidon 12 PF, and/or Kolloidon 17PF (BASF) at a concentration of about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, or about 10.0%.

In some embodiments, the high concentration low-viscosity formulation comprises Kolliphor HS 15 (BASF), Macrogol (15) hydroxystearate, polyethylene glycol (15)-hydroxystearate, polyoxyethylated 12-hydroxystearic acid, Solutol HS 15 at a concentration of about 0.1% to about 10%. In some embodiments, the high concentration low-viscosity formulation comprises Kolliphor HS 15 (BASF), Macrogol (15) hydroxystearate, polyethylene glycol (15)-hydroxystearate, polyoxyethylated 12-hydroxystearic acid, Solutol HS 15 at a concentration of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, or about 10.0%.

In some embodiments, the high concentration low-viscosity formulation comprises Benzyl alcohol at a concentration of about 0.1% to about 2.0%. In some embodiments, the high concentration low-viscosity formulation comprises Benzyl alcohol at a concentration of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0%.

In some embodiments the high concentration low-viscosity formulation comprises defibrotide in an amount between about 100 mg/mL to about 400 mg/mL, or about 150 mg/mL to about 250 mg/mL, or about 120 mg/mL to about 200 mg/mL, or about 180 mg/mL to about 200 mg/mL. In some embodiments, the high concentration low viscosity formulation comprises defibrotide at about 100 mg/mL, about 101 mg/mL, about 102 mg/mL, about 103 mg/mL, about 104 mg/mL, about 105 mg/mL, about 106 mg/mL, about 107 mg/mL, about 108 mg/mL, about 109 mg/mL, about 110 mg/mL, about 111 mg/mL, about 112 mg/mL, about 113 mg/mL, about 114 mg/mL, about 115 mg/mL, about 116 mg/mL, about 117 mg/mL, about 118 mg/mL, about 119 mg/mL, about 120 mg/mL, about 121 mg/mL, about 122 mg/mL, about 123 mg/mL, about 124 mg/mL, about 125 mg/mL, about 126 mg/mL, about 127 mg/mL, about 128 mg/mL, about 129 mg/mL, about 130 mg/mL, about 131 mg/mL, about 132 mg/mL, about 133 mg/mL, about 134 mg/mL, about 135 mg/mL, about 136 mg/mL, about 137 mg/mL, about 138 mg/mL, about 139 mg/mL, about 140 mg/mL, about 141 mg/mL, about 142 mg/mL, about 143 mg/mL, about 144 mg/mL, about 145 mg/mL, about 146 mg/mL, about 147 mg/mL, about 148 mg/mL, about 149 mg/mL, about 150 mg/mL, about 151 mg/mL, about 152 mg/mL, about 153 mg/mL, about 154 mg/mL, about 155 mg/mL, about 156 mg/mL, about 157 mg/mL, about 158 mg/mL, about 159 mg/mL, about 160 mg/mL, about 161 mg/mL, about 162 mg/mL, about 163 mg/mL, about 164 mg/mL, about 165 mg/mL, about 166 mg/mL, about 167 mg/mL, about 168 mg/mL, about 169 mg/mL, about 170 mg/mL, about 171 mg/mL, about 172 mg/mL, about 173 mg/mL, about 174 mg/mL, about 175 mg/mL, about 176 mg/mL, about 177 mg/mL, about 178 mg/mL, about 179 mg/mL, about 180 mg/mL, about 181 mg/mL, about 182 mg/mL, about 183 mg/mL, about 184 mg/mL, about 185 mg/mL, about 186 mg/mL, about 187 mg/mL, about 188 mg/mL, about 190 mg/mL, about 191 mg/mL, about 192 mg/mL, about 193 mg/mL, about 194 mg/mL, about 195 mg/mL, about 196 mg/mL, about 197 mg/mL, about 198 mg/mL, about 199 mg/mL, about 200 mg/mL, about 201 mg/mL, about 202 mg/mL, about 203 mg/mL, about 204 mg/mL, about 205 mg/mL, about 206 mg/mL, about 207 mg/mL, about 208 mg/mL, about 209 mg/mL, about 210 mg/mL, about 211 mg/mL, about 212 mg/mL, about 213 mg/mL, about 214 mg/mL, about 215 mg/mL, about 216 mg/mL, about 217 mg/mL, about 218 mg/mL, about 219 mg/mL, about 220 mg/mL, mg/mL, about 221 mg/mL, about 222 mg/mL, about 223 mg/mL, about 224 mg/mL, about 225 mg/mL, about 226 mg/mL, about 227 mg/mL, about 228 mg/mL, about 229 mg/mL, about 230 mg/mL, mg/mL, about 231 mg/mL, about 232 mg/mL, about 233 mg/mL, about 234 mg/mL, about 235 mg/mL, about 236 mg/mL, about 237 mg/mL, about 238 mg/mL, about 239 mg/mL, about 240 mg/mL, about 241 mg/mL, about 242 mg/mL, about 243 mg/mL, about 244 mg/mL, about 245 mg/mL, about 246 mg/mL, about 247 mg/mL, about 248 mg/mL, about 249 mg/mL, about 250 mg/mL, about 251 mg/mL, about 252 mg/mL, about 253 mg/mL, about 254 mg/mL, about 255 mg/mL, about 256 mg/mL, about 257 mg/mL, about 258 mg/mL, about 259 mg/mL, about 260 mg/mL, about 261 mg/mL, about 262 mg/mL, about 263 mg/mL, about 264 mg/mL, about 265 mg/mL, about 266 mg/mL, about 267 mg/mL, about 268 mg/mL, about 269 mg/mL, about 270 mg/mL, about 271 mg/mL, about 272 mg/mL, about 273 mg/mL, about 274 mg/mL, about 275 mg/mL, about 276 mg/mL, about 277 mg/mL, about 278 mg/mL, about 279 mg/mL, about 280 mg/mL, about 281 mg/mL, about 282 mg/mL, about 283 mg/mL, about 284 mg/mL, about 285 mg/mL, about 286 mg/mL, about 287 mg/mL, about 288 mg/mL, about 289 mg/mL, about 290 mg/mL, about 291 mg/mL, about 292 mg/mL, about 293 mg/mL, about 294 mg/mL, about 295 mg/mL, about 296 mg/mL, about 297 mg/mL, about 298 mg/mL, about 299 mg/mL, about 300 mg/mL about 301 mg/mL, about 302 mg/mL, about 303 mg/mL, about 304 mg/mL, about 305 mg/mL, about 306 mg/mL, about 307 mg/mL, about 308 mg/mL, about 309 mg/mL, about 310 mg/mL, about 311 mg/mL, about 312 mg/mL, about 313 mg/mL, about 314 mg/mL, about 315 mg/mL, about 316 mg/mL, about 317 mg/mL, about 318 mg/mL, about 319 mg/mL, about 320 mg/mL, about 321 mg/mL, about 322 mg/mL, about 323 mg/mL, about 324 mg/mL, about 325 mg/mL, about 326 mg/mL, about 327 mg/mL, about 328 mg/mL, about 329 mg/mL, about 330 mg/mL, about 331 mg/mL, about 332 mg/mL, about 333 mg/mL, about 334 mg/mL, about 335 mg/mL, about 336 mg/mL, about 337 mg/mL, about 338 mg/mL, about 339 mg/mL, about 340 mg/mL, mg/mL, 341 mg/mL, about 342 mg/mL, about 343 mg/mL, about 344 mg/mL, about 345 mg/mL, about 346 mg/mL, about 347 mg/mL, about 348 mg/mL, about 349 mg/mL, about 350 mg/mL, about 351 mg/mL, about 352 mg/mL, about 353 mg/mL, about 354 mg/mL, about 355 mg/mL, about 356 mg/mL, about 357 mg/mL, about 358 mg/mL, about 359 mg/mL, about 360 mg/mL, about 361 mg/mL, about 362 mg/mL, about 363 mg/mL, about 364 mg/mL, about 365 mg/mL, about 366 mg/mL, about 367 mg/mL, about 368 mg/mL, about 369 mg/mL, about 370 mg/mL, about 371 mg/mL, about 372 mg/mL, about 373 mg/mL, about 374 mg/mL, about 375 mg/mL, about 376 mg/mL, about 377 mg/mL, about 378 mg/mL, about 379 mg/mL, about 380 mg/mL, about 381 mg/mL, about 382 mg/mL, about 383 mg/mL, about 384 mg/mL, about 385 mg/mL, about 386 mg/mL, about 387 mg/mL, about 389 mg/mL, about 390 mg/mL, about 391 mg/mL, about 392 mg/mL, about 393 mg/mL, about 394 mg/mL, about 395 mg/mL, about 396 mg/mL, about 397 mg/mL, about 398 mg/mL, about 399 mg/mL, or about 400 mg/mL.

In some embodiments, the formulation comprises defibrotide in an amount between about 100 mg/mL to about 400 mg/mL, a viscosity reducer at a concentration of between about 5 mM and about 100 mM. In some embodiments, the viscosity reducer is glycylglycine, glycine, sodium citrate or benzyl alcohol. In some embodiments, the high concentration low viscosity formulation further comprises sodium citrate. In some embodiments, the sodium citrate is present at a concentration of between about 10 mM to about 34 mM.

In some embodiments, the high concentration defibrotide formulation further comprises between about 10 mM to about 34 mM sodium citrate. In some embodiments, the high concentration defibrotide formulation comprises about 180 mg/ml of defibrotide, about 10-100 mM viscosity reducer, and about 10-25 mM sodium citrate. In some embodiments, the high concentration defibrotide formulation comprises about 120-200 mg/mL of defibrotide, about 10-100 mM viscosity reducer, and about 10-25 mM sodium citrate, wherein the formulation is formulated for subcutaneous or intravenous delivery to a patient. In some embodiments, the viscosity reducer is glycylglycine, glycine, or sodium citrate.

Other excipients can be added to the present formulations, such as preservatives, salts, or pH adjusting agents.

In some embodiments of the disclosure, the viscosity of the low-viscosity formulation is between about 1 to about 70 cP. In some embodiments, the viscosity of the low-viscosity formulation is between about 5 cP to about 70 cP, or about 10 cP to about 65 cP. In some embodiments, the viscosity of the low-viscosity formulation is about 5 cP, about 10 cP, about 15 cP, about 20 cP, about 25 cP, about 30 cP, about 35 cP, about 40 cP, about 45 cP, about 50 cP, about 55 cP, about 60 cP, about 65 cP, or about 70 cP. In some embodiments, the viscosity of the low-viscosity formulation is about 1 cp, about 2 cp, about 3 cp, about 4 cp, about 5 cp, about 6 cp, about 7 cp, about 8 cp, about 9 cp, about 10 cp, about 11 cp, about 12 cp, about 13 cp, about 14 cp, about 15 cp, about 16 cp, about 17 cp, about 18 cp, about 19 cp, about 20 cp, about 21 cp, about 22 cp, about 23 cp, about 24 cp, about 25 cp, about 26 cp, about 27 cp, about 28 cp, about 29 cp, about 30 cp, about 31 cp, about 32 cp, about 33 cp, about 34 cp, about 35 cp, about 36 cp, about 37 cp, about 38 cp, about 39 cp, about 40 cp, about 41 cp, about 42 cp, about 43 cp, about 44 cp, about 45 cp, about 46 cp, about 47 cp, about 48 cp, about 49 cp, about 50 cp, about 51 cp, about 52 cp, about 53 cp, about 54 cp, about 55 cp, about 56 cp, about 57 cp, about 58 cp, about 59 cp, about 60 cp, about 61 cp, about 62 cp, about 63 cp, about 64 cp, about 65 cp, about 66 cp, about 67 cp, about 68 cp, about 69 cp, or about 70 cp.

In some embodiments, the high concentration low-viscosity formulation has a pKa value between about 5 to about 10. In some embodiments, the buffer or excipient has a pKa value of about 5, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, or about 10.0.

In certain embodiments, the high concentration low-viscosity formulation has a pH of about 6 to about pH 8. In some embodiments, the buffer or excipient has a pH of about 6.0, about 6.1, about, 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0.

In some embodiments of the disclosure, viscosity of the high concentration low-viscosity formulation decreases over time. In some embodiments, the viscosity decreases during storage of the formulation. In some embodiments, the viscosity of the high concentration low-viscosity formulation decreases over about 1 week to about 5 years. In some embodiments, the viscosity of the high concentration low-viscosity formulation decreases over about 1 week, about 2 weeks, about 3 weeks, about 4 weeks, about 5 weeks, about 6 weeks, about 7 weeks, about 8 weeks, about 9 weeks, about 10 weeks, about 12 weeks, about 13 weeks, about 15 weeks, about 16 weeks, about 17 weeks, about 18 weeks, about 19 weeks, about 20 weeks, about 5 months, about 6 months, about 7 months, about 8 months, about 9 months, about 10 months, about 11 months, about 1 year, about 2 years, about 3 years, about 4 years, or about 5 years.

In some embodiments, viscosity of the high concentration low-viscosity formulation decreases with an increase in temperature. In some embodiments, viscosity of the high concentration low-viscosity formulation decreases with increases in temperature of about 1° C. to about 50°. In some embodiments, viscosity of the high concentration low-viscosity formulation decreases with increases in temperature of about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C.

Testing of viscosity of the high concentration low-viscosity formulation is performed at ambient or room temperature (e.g from 15° C. to 35° C.). However, the high concentration low-viscosity formulation may be stored at any appropriate temperature. In some embodiments, the high concentration low-viscosity formulation is stored between 2° C. to about 60° C. In some embodiments, the high concentration low-viscosity formulation is stored at about 5±3° C., 25±2° C., room temperature, 40±2° C., or 60° C. In some embodiments, the high concentration low viscosity formulation is stored at about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., or about 60° C.

In some embodiments, viscosity of the high concentration low-viscosity formulation decreases when stored at a temperature of about 2° C. to about 60° C. In some embodiments, the viscosity of the high concentration low-viscosity formulation decreases when stored at about 5±3° C., 25±2° C., room temperature, 40±2° C., or 60° C. In some embodiments, the viscosity of the high concentration low-viscosity formulation decreases when stored at a temperature of about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., or about 60° C. In some embodiments, viscosity of the high concentration low viscosity formulation decreases when stored at room temperature (e.g. from 15° C. to 35° C.).

In some embodiments, the viscosity of the high concentration low-viscosity nucleic acid formulation provided herein decreases with decreasing mean molecular weight of the nucleic acid. In some embodiments, the viscosity of the high concentration low-viscosity nucleic acid formulation provided herein decreases with decreasing mean molecular weight of the nucleic acid at a given concentration of said nucleic acid. In some embodiments, the viscosity of the high concentration low-viscosity nucleic acid formulation provided herein decreases with decreasing mean molecular weight of the nucleic acid at a given concentration of said nucleic acid when viscosity is measured under room temperature conditions, such as from 15° C. to 35° C. In some embodiments, the viscosity of the high concentration defibrotide formulation decreases between about 50% to about 85% of its initial (time=T0) viscosity. In some embodiments, the viscosity of the high concentration defibrotide formulation decreases by about 50%, about 51%, about 52%, about 53%, about 54%, about 55%, about 56, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84% or about 85% of its initial (time=T0) viscosity.

In some embodiments, the viscosity decreases under increasing shear, agitation, and/or pressure. In some embodiments, the viscosity decreases during administration of the low-viscosity formulation (e.g. when passing through a needle). In some embodiments, the viscosity of the high concentration defibrotide formulation decreases between about 50% to about 85% of its initial (time=T0) viscosity. In some embodiments, the viscosity of the high concentration defibrotide formulation decreases by about 50%, about 51%, about 52%, about 53%, about 54%, about 55%, about 56, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84% or about 85% of its initial (time=T0) viscosity. In some embodiments, the determination of the viscosity of the low-viscosity formulation varies depending on the temperature at which it is measured.

In some embodiments, the viscosity of high concentration nucleic acid formulations provided herein increases with an increase in the mean molecular weight of the nucleic acid. In some embodiments, the viscosity of the high concentration defibrotide formulation increases between about 10% to about 100% of its initial (time=T0) viscosity. In some embodiments, the viscosity of the high concentration defibrotide formulation increases by about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84% about 85%, about 86%, about 87%, about 88%, about 89%, about 90%, about 91%, about 92%, about 93%, about 94%, about 95%, about 96%, about 97%, about 98%, about 99%, about 100% of its initial (time=T0) viscosity.

In preferred embodiments, the viscosity is measured under room temperature conditions, such as from 15° C. to 35° C. More preferably, the viscosity is measured between 18° C. to 25° C. Even more preferably, the viscosity is measured at between 21° C. to 23° C.

In some embodiments, the high concentration low-viscosity formulations of the present disclosure have an osmolality between about 200 mOsm/kg and about 1000 mOsm/kg. In some embodiments, the low-viscosity formulations of the present disclosure have an osmolality between about 240 mOsm/kg to about 600 mOsm/kg or about 300 mOsm/kg to about 550 mOsm/kg. In some embodiments, the low-viscosity formulations of the present disclosure have an osmolality of about 200 mOsm/kg, about 210 mOsm/kg, about 220 mOsm/kg, about 230 mOsm/kg, about 240 mOsm/kg, about 250 mOsm/kg, about 260 mOsm/kg, about 270 mOsm/kg, about 280 mOsm/kg, about 290 mOsm/kg, about 300 mOsm/kg, about 310 mOsm/kg, about 320 mOsm/kg, about 330 mOsm/kg, about 340 mOsm/kg, about 350 mOsm/kg, about 360 mOsm/kg, about 370 mOsm/kg, about 380 mOsm/kg, about 390 mOsm/kg, about 400 mOsm/kg, about 410 mOsm/kg, about 420 mOsm/kg, about 430 mOsm/kg, about 440 mOsm/kg, about 450 mOsm/kg, about 460 mOsm/kg, about 470 mOsm/kg, about 480 mOsm/kg, about 490 mOsm/kg, about 500 mOsm/kg, about 510 mOsm/kg, about 520 mOsm/kg, about 530 mOsm/kg, about 540 mOsm/kg, about 550 mOsm/kg, about 560 mOsm/kg, about 570 mOsm/kg, about 580 mOsm/kg, about 590 mOsm/kg, about 600 mOsm/kg, about 610 mOsm/kg, about 620 mOsm/kg, about 630 mOsm/kg, about 640 mOsm/kg, about 650 mOsm/kg, about 660 mOsm/kg, about 670 mOsm/kg, about 680 mOsm/kg, about 690 mOsm/kg, about 700 mOsm/kg, about 710 mOsm/kg, about 720 mOsm/kg, about 730 mOsm/kg, about 740 mOsm/kg, about 750 mOsm/kg, about 760 mOsm/kg, about 770 mOsm/kg, about 780 mOsm/kg, about 790 mOsm/kg, about 800 mOsm/kg, about 810 mOsm/kg, about 820 mOsm/kg, about 830 mOsm/kg, about 840 mOsm/kg, about 850 mOsm/kg, about 860 mOsm/kg, about 870 mOsm/kg, about 880 mOsm/kg, about 890 mOsm/kg, about 900 mOsm/kg, about 910 mOsm/kg, about 920 mOsm/kg, about 930 mOsm/kg, about 940 mOsm/kg, about 950 mOsm/kg, about 960 mOsm/kg, about 970 mOsm/kg, about 980 mOsm/kg, about 990 mOsm/kg, or about 1000 mOsm/kg.

In certain embodiments, the viscosity of the high concentration defibrotide formulation decreases during storage up to about 85% of its initial (time=T0) viscosity under room temperature storage conditions. In some embodiments, the viscosity of the high concentration defibrotide formulation decreases during storage between about 50% to about 85% of its initial (time=T0) viscosity under room temperature storage conditions. In some embodiments, the viscosity of the high concentration defibrotide formulation decreases during storage by about 50%, about 51%, about 52%, about 53%, about 54%, about 55%, about 56, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84% or about 85% of its initial (time=T0) viscosity under room temperature storage conditions.

In some embodiments, the high concentration low-viscosity formulation comprises between 100 mg/mL to about 400 mg/mL of defibrotide, and a viscosity reducer at a concentration of between about 5 mM and about 100 mM, wherein the formulation has a viscosity between about 5 and about 70 cP when measured at between 15° C. and 25° C., and an osmolality between about 240 mOsm/kg and about 1000 mOsm/kg. In some embodiments, the viscosity reducer is glycylglycine, glycine, sodium citrate, benzyl alcohol or a hyaluronidase (e.g. PH20). In some embodiments, the high concentration low viscosity formulation further comprises sodium citrate. In some embodiments, the sodium citrate is present at a concentration of between about 10 mM to about 34 mM.

In some embodiments, the present disclosure provides for methods for delivering the formulations of the disclosure. In certain embodiments, the formulations of the present disclosure are subcutaneously delivered. In some embodiments, formulations of the disclosure are administered subcutaneously by means of an automated injection device as disclosed herein that can be used by the patient. In some embodiments, the high concentration low-viscosity formulation is a defibrotide formulation. In some embodiments, the formulation is a High Concentration Liquid Formulation (HCLF).

Devices for subcutaneous administration may be prefilled, with for example a predefined adult or pediatric dose, or may be used to administer a weight-based dose specific for individual patients. In some embodiments, the patient determines the dose and administers it. In some specific embodiments, formulations of the disclosure are administered subcutaneously in less than about two hours, less than about one hour, or less than about 30 minutes. In some specific embodiments, formulations of the disclosure are delivered subcutaneously over about 5 minutes to about 6 hours, about 10 minutes to about 1 hour or about 15 minutes to about 45 minutes. In some embodiments, the high concentration low-viscosity formulations are delivered subcutaneously over about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes, about 160 minutes, about 165 minutes, about 170 minutes, about 175 minutes, about 180 minutes, about 185 minutes, about 190 minutes, about 200 minutes, about 205 minutes, about 210 minutes, about 215 minutes, about 220 minutes, about 225 minutes, about 230 minutes, about 235 minutes, about 240 minutes, about 245 minutes, about 250 minutes, about 260 minutes, about 265 minutes, about 270 minutes, about 275 minutes, about 280 minutes, about 285 minutes, about 290 minutes, about 295 minutes, about 300 minutes, about 305 minutes, about 310 minutes, about 315 minutes, about 320 minutes, about 325 minutes, about 330 minutes, about 335 minutes, about 340 minutes, about 345 minutes, about 350 minutes, about 355 minutes, or about 360 minutes. In some embodiments, the infusion delivers a consistent concentration of the high concentration low viscosity formulation. In some embodiments, the infusion delivers a varied concentration of the high concentration low-viscosity formulation. In some embodiments, the initial concentration of the high concentration low-viscosity formulation is higher than the concentration delivered later in the regimen. In some embodiments, the initial concentration of the high concentration low-viscosity formulation is lower than the concentration delivered later in the regimen. In some embodiments, a bolus of the high concentration low-viscosity formulation is delivered before infusion of the same formulation begins. In some embodiments, the concentration of the high concentration low-viscosity formulation administered via bolus is greater than the concentration of the high concentration low-viscosity formulation administered via infusion.

In some embodiments, the high concentration low-viscosity formulation given subcutaneously exhibits improved Cmax and/or AUC compared to an intravenously administered defibrotide formulation. In some embodiments, the high concentration low-viscosity formulation administered subcutaneously exhibits a Cmax about 5% to about 500% greater than the Cmax exhibited by intravenously administering the high concentration low-viscosity formulation. In some embodiments, the high concentration low-viscosity formulation administered subcutaneously exhibits Cmax about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84% about 85%, about 86%, about 87%, about 88%, about 89%, about 90%, about 91%, about 92%, about 93%, about 94%, about 95%, about 96%, about 97%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% or more greater than the Cmax exhibited by intravenously administering the high concentration low-viscosity formulation.

In some embodiments, the high concentration low-viscosity formulation administered subcutaneously exhibits a AUC about 5% to about 500% greater than the AUC exhibited by intravenously administering the high concentration low-viscosity formulation. In some embodiments, the high concentration low-viscosity formulation administered subcutaneously exhibits AUC about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84% about 85%, about 86%, about 87%, about 88%, about 89%, about 90%, about 91%, about 92%, about 93%, about 94%, about 95%, about 96%, about 97%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% or more greater than the AUC exhibited by intravenously administering the high concentration low-viscosity formulation.

The formulation dosing may be determined by a variety of factors that will be readily apparent to a skilled artisan. In some embodiments, the dose is based on patient's baseline body weight. In some embodiments, formulation is administered in an amount of about 1 to about 100 mg per kilogram of body weight per day. For example the formulation is administered in an amount of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg per kilogram of body weight per day. In some embodiments, formulation is administered in an amount of about 25 mg per kilogram of body weight per day. In some embodiments, doses based on the patient's body weight are rounded to the nearest 10 mg for patients over 35 kg. In some embodiments, doses based on the patient's body weight were rounded to the nearest 5 mg for patients under 35 kg. In some embodiments, the formulation is a defibrotide formulation.

The formulation may be administered as a single daily dose or in multiple doses per day. In some embodiments, formulation is administered once a day. In some embodiments, formulation is administered in multiple doses per day. For example, the formulation may be administered in 2, 3, 4, 5, 6, 7, 8, 9, or in 10 doses per day. In some embodiments, the formulation is administered in four doses per day. In some embodiments, the formulation is administered in four doses per day every 6 hours.

In some embodiments, subcutaneous administration of the low-viscosity formulations of the present disclosure allows for less-frequent administration and/or lower doses. In some embodiments, subcutaneous administration of the low-viscosity formulation of the present disclosure allows for reduced administration volume.

As a skilled artisan will appreciate, the treatment period may vary on a patient-by-patient basis. In some embodiments, the treatment period is determined by monitoring signs and symptoms of the disease being treated. In some embodiments, the high concentration low-viscosity formulations of the present disclosure are administered until patient signs and symptoms of the disease being treated are decreased, ameliorated, delayed, or treated. In some embodiments, the high concentration low-viscosity formulations of the present disclosure are administered until patient signs and symptoms of the disease being treated are decreased, ameliorated, delayed by about 1% to about 100% compared to the signs and symptoms of the disease in the same patient before treatment or an untreated patient. In some embodiments, the high concentration low-viscosity formulations of the present disclosure are administered until patient signs and symptoms of the disease being treated are decreased, ameliorated, delayed by at least about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100% compared to the signs and symptoms of the disease in the same patient before treatment or an untreated patient. For example, in some embodiments, the treatment period is determined by monitoring signs and symptoms of hepatic VOD. For example, if the signs and symptoms of hepatic VOD are still present after an initial treatment period, defibrotide treatment is continued until resolution of VOD. In some embodiments, if the signs and symptoms of hepatic VOD are still present after 21 days, defibrotide treatment is continued until resolution of VOD up to a maximum of 60 days. Thus, in certain embodiments, the treatment period may last anywhere from 21 to 60 days. For example, the treatment period lasts for 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the treatment period lasts 21 days.

In some embodiments, administration of the formulations of the present disclosure treats or ameliorates development of VOD and/or VOD symptoms compared to an untreated patient or the same patient before formulation administration. In some embodiments, VOD and/or VOD symptoms are treated or ameliorated in the patient between day 1 and year 10. In some embodiments, administration of the formulation treats or ameliorates development of VOD and/or VOD symptoms compared to an untreated patient or the same patient before defibrotide administration at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, administration of the formulation treats or ameliorates development of VOD and/or VOD symptoms compared to an untreated patient or the same patient before formulation administration for about 1 day, about 1 week, about 1 month, about 2 months, about 3 months, about 4 months, about 5 months, about 6 months, about 1 year, about 2 years, about 5 years, or about 10 years, or more.

In some embodiments, administration of the formulations of the present invention prevents VOD and/or VOD symptoms.

In some embodiments, administration of the formulations of the present disclosure treats or ameliorates VOD and/or VOD symptoms by about 1%, about 5%, about 10%, about 20%, about 30%, about 40%, about 50%, about 60%, about 70%, about 80%, about 90%, or about 100% compared to an untreated patient or the same patient before formulation administration. In some embodiments, administration of the formulation treats or ameliorates development of VOD and/or VOD symptoms compared to an untreated patient or the same patient before formulation administration by about 1%, about 5%, about 10%, about 20%, about 30%, about 40%, about 50%, about 60%, about 70%, about 80%, about 90%, or about 100% at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, administration of the formulation treats or ameliorates development of VOD and/or VOD symptoms compared to an untreated patient or the same patient before formulation administration by about 1%, about 5%, about 10%, about 20%, about 30%, about 40%, about 50%, about 60%, about 70%, about 80%, about 90%, or about 100% for about 1, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years or more.

Automated Injection Device

The automated injection devices described herein may be useful in preparing and/or administering the high concentration low-viscosity defibrotide formulations mentioned above to a patient, and may be part of a system configured to prepare the formulations, for example, mix the formulations and/or transfer the formulations, etc., to the injection device. Some aspects of the systems and automated injection devices are disclosed in U.S. Pat. No. 9,925,333, the contents of which are herein incorporated by reference in their entirety for all purposes. The automated injection devices of the instant disclosure may be beneficial when subcutaneously administering a therapeutically effective amount of the high concentration defibrotide formulations described herein. The automated injection devices of the instant disclosure may be beneficial for administering any therapeutically effective amount of defibrotide. In some embodiments, the defibrotide formulation is a high concentration defibrotide formulation including a high concentration low-viscosity defibrotide formulation as described herein. In some embodiments, the automated injection device cannula diameter is configured to provide a flow rate suitable for delivering a high concentration defibrotide formulation. For example, the cannula diameter may be sized for subcutaneous delivery of a high concentration formulation.

In some embodiments, the present disclosure provides an automated injection device that is a disposable, one-time-use device that may be part of a system that allows for the manual transfer of injectable defibrotide components from one or more standard vials into an injection device and preferably simultaneously pressurizes the injection device for subsequent automated injection into a patient.

In some embodiments, manual transfer of the injectable defibrotide into the injection device is done using a syringe. In some embodiments, manual transfer of the injectable defibrotide into the injection device is performed by the patient and through use of a syringe. Other means of transferring the defibrotide into the injection device may also be used, such as those described in U.S. Pat. No. 9,925,333 incorporated herein by reference.

The injection devices disclosed herein may employ an expandable member, such as a balloon or bladder, to automatically expel or inject the formulation when activated by the patient. Long term storage of a defibrotide formulation in a pressurized member may present design and manufacturing challenges. However, one beneficial aspect of the injection devices and methods disclosed herein is that the expandable member may remain unpressurized (e.g., the balloon unfilled and unexpanded and in a low energy state) and the defibrotide contained in its standard original vial or vials for enhanced shelf life until an injection is required. At that time, the defibrotide may be transferred, e.g. manually transferred or using a transfer apparatus, from a vial or vials into the injection device (with any associated mixing, diluting or other processing as required). When a transfer apparatus is employed, the transfer apparatus may simultaneously charge the injection device (e.g., expand and pressurize the expandable member or balloon by introducing the defibrotide into it under pressure) so that the injection device is ready for automated injection into a patient upon user activation. Given that the high concentration defibrotide formulation is in the injection device only for a very limited amount of time, such as seconds or minutes, and shelf life concerns and design or material constraints for long term drug storage are reduced.

In some embodiments, the expandable member (such as a balloon or bladder) may be elongated and configured to progressively collapse from one end to another during injection. The specific configuration may vary, but arrangement of the elongated expandable member in a generally flat spiral or helical configuration may allow for the expandable member to be of substantial length and volume in a relatively compact arrangement that can be applied to and retained on the skin of a patient during injection. The injection device may also have a viewing window that allows the patient to view the expandable member and identify the general status of the injection by the amount of collapse and/or the expandable member, or the viewing window may be graduated by appropriate markings so that the patient can determine the amount of defibrotide injection that has occurred.

The automated injection devices described herein are generally configured so that a patient can self-inject high concentration low viscosity defibrotide formulations. The automated injection devices may include a housing defining a central portion, where the housing has a circular profile and a spiral recess. A bladder may be positioned within the housing, where the bladder includes a first end, a second end, and a wall extending between the first end and the second end. The bladder may be expandable, and when positioned within the spiral recess of the housing, configured to move along the spiral recess upon contraction and expansion thereof. The bladder wall may include an area of increased thickness along a first portion thereof, where the area of increased thickness aids in deflecting at least a second portion of the bladder wall during formulation administration to effect the movement of the distal end of the bladder along an arcuate path.

A cannula may be disposed within the central portion of the housing and configured to be movable between multiple positions, such as a pre-dispense position where the cannula is not in fluid communication with the bladder, and a dispense position where the cannula is in fluid flow communication with the bladder. The cannula may have an inner diameter ranging from about 0.08 mm to about 0.41 mm, or from about 0.10 mm to about 0.30 mm. In some embodiments, the cannula inner diameter may be about 0.08 mm, about 0.10 mm, about 0.12 mm, about 0.13 mm, about 0.14 mm, about 0.15 mm, about 0.16 mm, about 0.17 mm, about 0.18 mm, about 0.19 mm, about 0.20 mm, about 0.21 mm, about 0.22 mm, about 0.23 mm, about 0.24 mm, about 0.25 mm, about 0.26 mm, about 0.27 mm, about 0.29 mm, about 0.30 mm, about 0.31 mm, about 0.35 mm, about 0.40, about 0.41 mm, or 0.50 mm. In one embodiment, it may be useful for the cannula inner diameter to be about 0.17 mm. In another embodiment, it may be useful for the cannula inner diameter to be about 0.27 mm. The cannula may also include a side hole configured so that when the cannula is in the pre-dispense or a first depth position with respect to the surface of the skin of a patient, the side hole is not in fluid communication with the bladder, and when the cannula is in the dispense position or a second depth position with respect to the surface of the patient's skin, the side hole is in fluid flow communication with the bladder. Here the second depth position is closer to the patient's skin surface than the first depth position.

The injection devices may further include a cannula actuator coupled to the cannula of the injection device, where the high concentration low viscosity formulations (e.g., high concentration low-viscosity formulations of defibrotide) are administered by movement of the cannula from the pre-dispense position to i) the first depth position; and ii) to the second depth position; and then 3) retention of the injection cannula at the second depth position.

The injection devices may additionally include a manifold disposed within the housing and coupled to the first end of the bladder, where the manifold comprises a fluid inlet port configured to receive a high concentration low viscosity formulation and a fluid pathway configured for fluid communication between the fluid inlet port and the bladder for introduction of the high concentration low viscosity formulation under pressure into the bladder. The high concentration low viscosity formulation may contain defibrotide.

Furthermore, a septum positioned within the housing may be provided and configured to seal around a periphery of the side hole of the cannula when the cannula is in the pre-dispense position or in the first depth position. An indicator disposed on the second end of the bladder may provide an indication of dosing progress to the patient; and a mandrel coupled to the second end of the bladder may be used to pre-stress the bladder to an expanded condition.

To attach the injection device to the patient, an adhesive may be disposed on the device housing. Once attached, the injection device may deliver a high concentration low-viscosity defibrotide formulation to the patient. For example, a high concentration defibrotide formulation comprising between 100 mg/mL to about 400 mg/mL of defibrotide, and viscosity reducer at a concentration of between about 5 mM and about 100 mM may be delivered, where the formulation has a viscosity between about 5 and about 70 cP when measured at a temperature of between 15° C. and 25° C., and an osmolality between about 240 mOsm/kg and about 1000 mOsm/kg. Preferably, the viscosity reducer is glycylglycine, glycine, sodium citrate, benzyl alcohol or hyaluronidase (PH20). The high concentration low viscosity formulation may be delivered parenterally to the patient, for example, subcutaneously. In some embodiments, the high concentration low viscosity formulation is delivered via the subcutaneous route. The flow rate of the formulation through the cannula may be between about 0.5 ml/hour, about 1 ml/hour, about 5 ml/hour, about 10 ml/hour to about 200 ml/hour or between about 15 ml/hour to about 150 ml/hour. In some embodiments, the flow rate may be about 10 ml/hour, about 15 ml/hour, about 18 ml/hour, about 20 ml/hour, about 30 ml/hour, about 40 ml/hour, about 50 ml/hour, about 60 ml/hour, about 70 ml/hour, about 80 ml/hour, about 90 ml/hour, about 100 ml/hour, about 110 ml/hour, about 120 ml/hour, about 130 ml/hour, about 140 ml/hour, about 145 ml/hour, about 148 ml/hour, about 150 ml/hour, about 160 ml/hour, about 170 ml/hour, about 180 ml/hour, about 190 ml/hour, about 200 ml/hour, about 250 ml/hour, about 300 ml/hour, about 350 ml/hour, about 400 ml/hour, about 450 ml/hour, about 500 ml/hour, about 550 ml/hour, about 600 ml/hour, or about 700 ml/hour. In one embodiment, it may be useful for the cannula flow rate to be about 15 ml/hour to about 35 ml/hour. In one embodiment, it may be useful for the cannula flow rate to be about 18 ml/hour. In another embodiment, it may be useful for the cannula flow rate to be about 148 ml/hour. For example, when a cannula flow rate of about 18 ml/hour is desired for the subcutaneous injection device, the cannula may have an inner diameter of about 0.17 mm. Further, when a cannula flow rate of about 148 ml/hour is desired for the subcutaneous injection device, the cannula may have an inner diameter of about 0.27 mm.

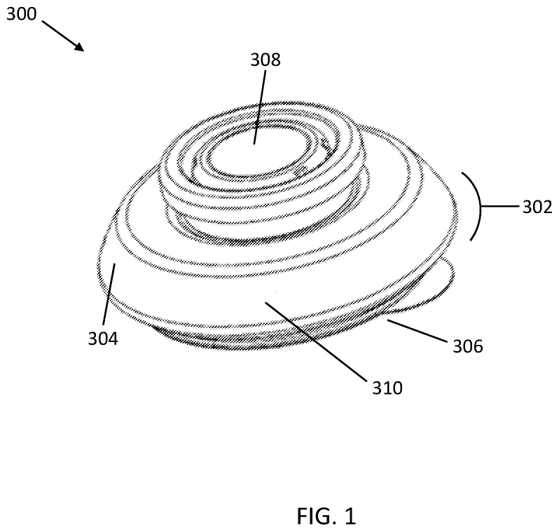

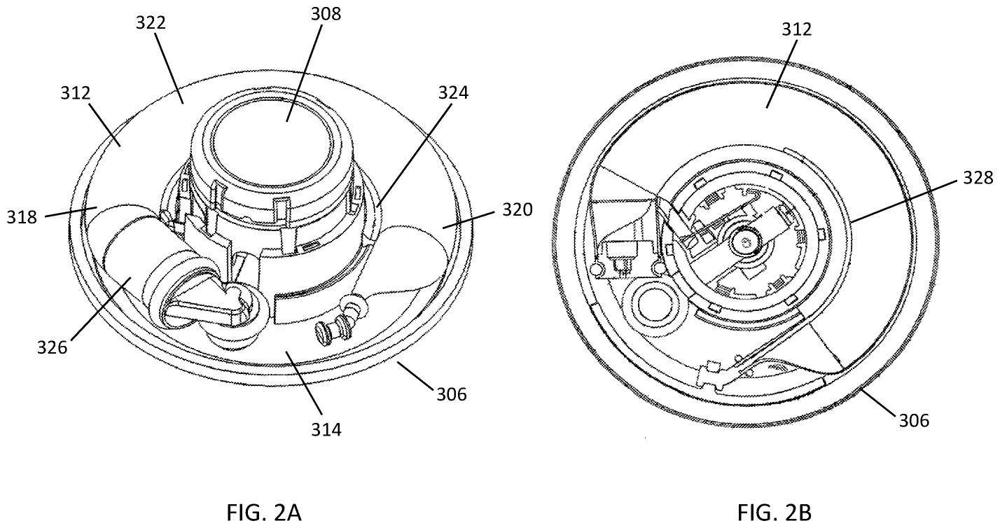

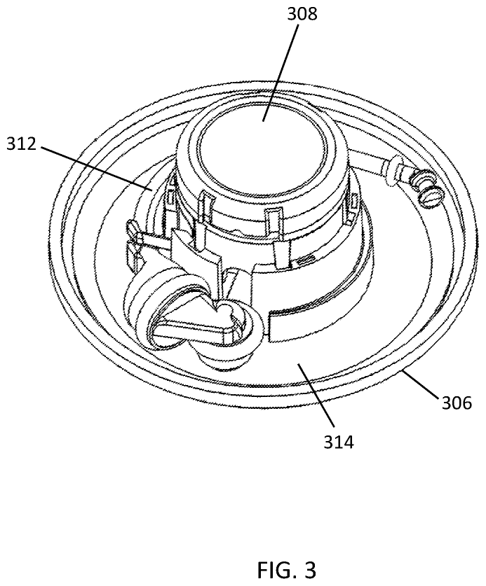

In some embodiments, the housing defining a central portion has a circular profile. In some embodiments, the housing defining a central portion includes a spiral recess within which a second end of a bladder is positioned and moves along upon contraction and expansion. For example, as shown in FIGS. 1 and 2A, the injection device (300) has a generally low-profile, circular shaped housing (302) having an upper surface (304) and a lower surface (306), through which a cannula (not shown) protrudes when actuated by a patient. The upper surface (304) has an actuator or button (308) that is depressed to start the injection. A window (310) of the housing (302) allows the patient or a medical professional to view an expandable member, bladder (312), within the housing (302) and ascertain the amount of the high concentration low viscosity defibrotide formulation in the injection device (300). Providing the window (310) may help the patient determine whether the injection has commenced or concluded. In some embodiments, the expandable member (312) and/or the window (310) may be graduated, such as by line markings or the like, so that the patient or medical professional can visually determine the amount of the defibrotide formulation remaining with greater precision, and thus, visually determine whether about 50% or about 75% of the defibrotide formulation, etc., has been delivered. In addition, the bladder (312) may itself include or interact with a feature on the housing (302) to show the amount of the defibrotide formulation remaining. For example, when the injection device (300) is full of the defibrotide formulation, the window (310) may show one color such as but not limited to green. When the injection device (300) is empty of defibrotide formulation, as shown in FIG. 3, the window (310) may show a different color such as but not limited to red. Accordingly, in the middle of dispense, the window (310) could show a combination of colors.

The bladder (312) employed in the injection device (300) may take the form of an elongated bladder arranged, for example, in a planar helical or spiral configuration as illustrated in FIG. 2A. As previously mentioned, the injection device (300) includes a circular shaped housing (302). The circular shaped housing (302) may comprise a spiral slot or recess (314) formed therein. The elongated bladder (312) rests in the spiral recess (314), with one end (e.g., proximal end (318)) for communicating directly or indirectly with a cannula (not shown). The elongated spiral configuration may allow the bladder to have substantial volume for such quantity of defibrotide formulation as may be desired, while also contributing to the low-profile configuration of the injection device. In other words, by utilizing a relatively long expandable member with a large length to diameter ratio, very high pressures and volumes can be achieve with a minimum of forces required. Additionally, the volume of the expandable member can be changed by changing the filling length, without significantly altering the pressure/volume curves of the expandable member.

In some embodiments, the bladder positioned within the housing includes a first end, a second end, and a wall extending between the first end and the second end. In some embodiments, the bladder is expandable. In some embodiments the bladder expands in an arcuate manner. There are a number of different ways to cause an expandable member such as a bladder to expand and/or contract in an arcuate manner. For example, one way is to design the expandable member with a thicker wall cross section in one area around the circumference of the expandable member that would cause the expandable member to expand in a circular fashion. Alternatively, a separate element could be affixed along the length of the expandable member to effectively stiffen the expandable member in that portion of the circumference that would cause the expandable member to expand in an arcuate manner. Another way is to use internal features such as slots or recesses in the housing of the injection device to guide the expandable member around a circular or spiral path. These features could interact with the expandable member in a number of ways, the simplest being that the outer shape of the expandable member is constrained by a slot in the housing of the injection device. Friction between the expandable member and the inner surfaces of the housing could be reduced by lubricating the outside surface of the expandable member, or by inserting the expandable member within a low spring rate spring that would limit both the friction and outer diameter of the expandable member while not constraining the length.

The elongated expandable member may be preferably configured to expand along an arc with a predetermined tube diameter without the aid of walls or a guide within the injection device. Here a thicker wall area in a small portion of the circumference of the expandable member may be added to cause the elongated expandable member to expand in an arc as previously described. The arcuate expandable member grows in length due to increase in pressure and volume there within; the thicker section deflects less than the thinner section.

In some embodiments, the bladder wall includes an area of increased thickness along a side of the bladder wall, where the area of increased thickness deflects less than a remaining portion of the bladder wall during administration to affect the movement of the distal end of the bladder along an arcuate path. For example, referring to FIG. 2A, bladder (312) has a proximal end or first end (318) coupled to a manifold (326), a distal end or second end (320), and a wall (322) extending therebetween. The bladder (312) will expand in length in an arc shape in a manner that orients its increased wall thickness zone (324) or less deflecting zone to the inside of the circle. Increasing the wall thickness of the bladder (312) within the small zone (324) around the circumference will effectively continue to decrease the radius of the arc of the bladder (312). The zone of increased wall thickness may be achieved by molding or extruding it into the bladder or by bonding a strip of material to one side of the bladder to cause that portion of the wall to lengthen at a slower rate, thereby causing the bladder to expand in an arc shape. Proximal end (318) may also be a proximal exit port end of the expandable member (312).

Alternately, the expandable member could be pre-stretched and flattened around a circular diameter inside the injection device such as wall so that there would be no change in expandable member length. Alternatively, a straight or curved mandrel whose length is more than the unstressed expandable member could be used to stretch the expandable member into a circular shape within the injection device prior to filling. Alternatively, a mandrel could be used as a visual indicator to show the state of the injection device and the progress of the injection. The mandrel could be colored to allow it to be easily viewed through the housing.

In some embodiments, the automated injection device includes a cannula disposed within the central portion of the housing. In some embodiments, the cannula is configured to be movable between multiple positions. In some embodiments, the multiple positions include a pre-dispense position where the cannula is not in fluid communication with the bladder, and a dispense position where the cannula is in fluid flow communication with the bladder.

In some embodiments, the cannula diameter is configured for a specific high concentration defibrotide formulation. In some embodiments, the cannula diameter is configured to deliver a high concentration defibrotide formulation at a particular flow rate. In some embodiments, the cannula diameter ranges from about 0.10 mm to about 0.30 mm, or from about 0.15 mm to about 0.30 mm. In some embodiments, the cannula inner diameter may be about 0.10 mm, about 0.12 mm, about 0.13 mm, about 0.14 mm, about 0.15 mm, about 0.16 mm, about 0.17 mm, about 0.18 mm, about 0.19 mm, about 0.20 mm, about 0.21 mm, about 0.22 mm, about 0.23 mm, about 0.24 mm, about 0.25 mm, about 0.26 mm, about 0.27 mm, about 0.29 mm, or about 0.30 mm. In one embodiment, it may be useful for the cannula inner diameter to be about 0.17 mm. In another embodiment, it may be useful for the cannula inner diameter to be about 0.27 mm. In some embodiments, the defibrotide formulation flow rate resulting from the aforementioned inner cannula diameters is between about 10 ml/hour to about 200 ml/hour, or between about 15 ml/hour to about 150 ml/hour. In some embodiments, the flow rate may be about 10 ml/hour, about 15 ml/hour, about 18 ml/hour, about 20 ml/hour, about 30 ml/hour, about 40 ml/hour, about 50 ml/hour, about 60 ml/hour, about 70 ml/hour, about 80 ml/hour, about 90 ml/hour, about 100 ml/hour, about 110 ml/hour, about 120 ml/hour, about 130 ml/hour, about 140 ml/hour, about 145 ml/hour, about 148 ml/hour, about 150 ml/hour, about 160 ml/hour, about 170 ml/hour, about 180 ml/hour, about 190 ml/hour, or about 200 ml/hour. In one embodiment, it may be useful for the cannula flow rate to be about 18 ml/hour. In another embodiment, it may be useful for the cannula flow rate to be about 148 ml/hour. In one embodiment, when the cannula inner diameter is about 0.17 mm, the flow rate is about 18 ml/hour. In another embodiment, when the cannula inner diameter is about 0.27 mm, the flow rate is about 148 ml/hour. In some embodiments, the defibrotide formulation is a high concentration, low-viscosity defibrotide formulation.

In some embodiments, the cannula includes a side hole. In some embodiments, the side hole is configured so that when the cannula is in the pre-dispense position or in a first depth position, the side hole is not in fluid communication with the bladder, and when the injection cannula is in a second depth position, the side hole is in fluid flow communication with the bladder.

In some embodiments, the cannula of the injection device is coupled to a cannula actuator. This cannula actuator administers the defibrotide formulation by movement of the cannula from the pre-dispense position to i) a first depth position into a patient's skin, and ii) to a second depth position less than the first depth and the retention of the injection cannula at the second depth.

In some embodiments, the housing includes a manifold disposed within it. In some embodiments, the manifold is coupled to the first end of the bladder. In some embodiments, the manifold includes a fluid inlet port configured to receive a high concentration defibrotide formulation, and a fluid pathway configured for fluid communication between the fluid inlet port and the bladder for introduction of the high concentration defibrotide formulation under pressure into the bladder. In some embodiments, the second end of the bladder is moveable within the housing. In some embodiments, the bladder is configured to elongate between the first end and the second end.

In some embodiments, the housing contains a septum positioned within it. In some embodiments, the septum is configured to seal around a periphery of the side hole of the cannula when the injection cannula is in the pre-dispense position or in the first-depth position.

In some embodiments, the automated injection device includes an indicator. In some embodiments, the indicator is disposed on the second end of the bladder. In some embodiments, the indicator provides an indication of dosing progress to the patient.

In some embodiments, the automated injection device includes a mandrel coupled to the second end of the bladder and configured so that the bladder is pre-stressed to an expanded condition even after all high concentration defibrotide formulation has been expelled from the bladder.

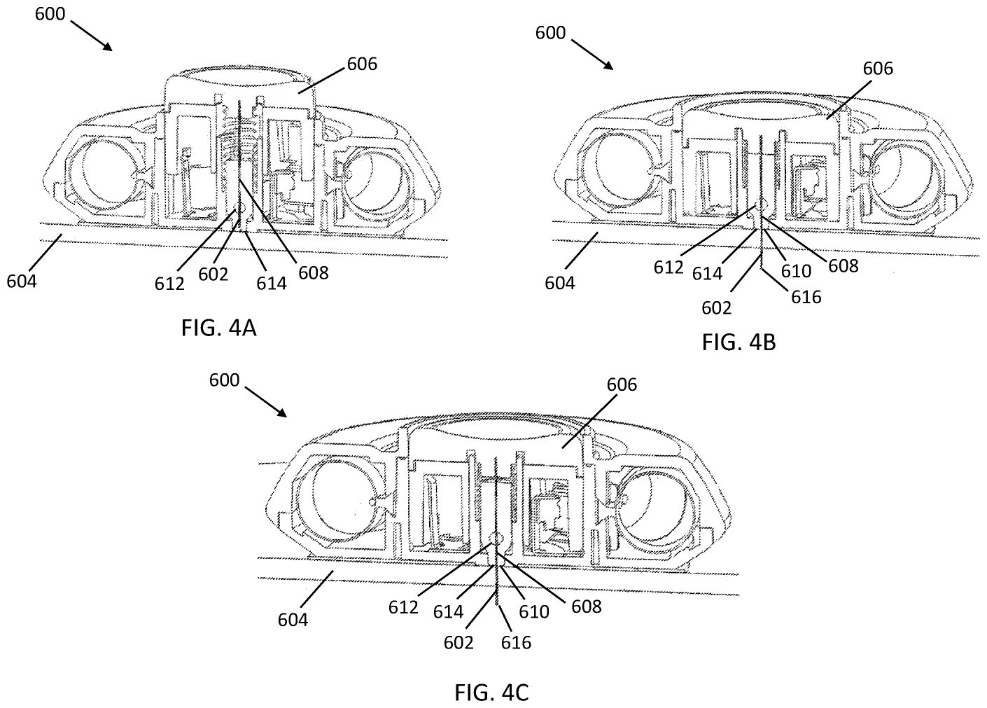

Referring to FIGS. 4A-4C, the injection device (600) may have a cannula (602) travel into the skin (604) of the patient, upon actuation of the button (606). Upon button actuation, the cannula (602) moves from a pre-fire, retracted state (FIG. 4A) to a first position or depth as shown in FIG. 4B and retracts slightly to a second position or depth preferably automatically as shown in FIG. 4C. The first depth position shown may be achieved from over travel of the button (606) during actuation. In general, the second depth position will be closer to the patient's skin surface than the first depth position. The final depth (e.g., the second depth position) of the cannula may be suitable for subcutaneous injections. Upon reaching the first depth position, the cannula may retract back to a second depth position. The retraction distance of the cannula to the second depth position may be in the range of about 0.1-2 mm. This retraction feature may be preferable to prevent the cannula from being blocked by tissue during the initial insertion process. This tissue blockage could require a very high pressure to overcome and prevent the injection device from delivering the high concentration low viscosity defibrotide formulations. The retraction of the cannula from the first depth position to the second depth position may create an open pocket ahead of the cannula tip allowing reduced pressure for initiation of flow of the defibrotide formulation from the cannula. This reduced pressure for initiation of the flow of the defibrotide formulation from the cannula is preferable for the injection device to maintain a relatively constant pressure during injection.

Again referring to FIGS. 4A-4C, the injection device (600) may also include a cannula (602) comprising a side hole (608). As shown in FIG. 4B, once the button (606) on the injection device (600) is fully depressed, the cannula (602) will be fully inserted into the skin (604) through a dispense port (610) and the injection device (600) will begin dispensing of the defibrotide formulation. Until the button (606) is fully depressed, the side-hole (608) and therefore the internal lumen of the cannula (602) is not in communication with the fluid channel (612) of the dispense port (610). Both the side-hole (608) and cannula tip (616) are retained within a septum (614). With the side-hole (608) and cannula tip (616) being retained within the septum (614), the entire defibrotide formulation path is kept sterile until the time of use. When the button (606) is fully depressed and the cannula (602) is in the dispense position, the side hole (608) in the cannula (602) communicates with the fluid channel (612) of the dispense port (610) so that injection of the defibrotide formulation can begin.

The septum may further provide the advantage of sealing the cannula tip as well as the side hole from the defibrotide formulation before and after dispense. Sealing the cannula tip and the side hole of the cannula at the end of the injection has a particular advantage to prevent dripping of defibrotide formulation from the injection device after end of dispense and/or after it is removed from the skin surface. It also prevents contaminates from entering the hollow cannula prior to being actuated into the skin. The septum may be made of any suitable material to allow for sealing once the cannula has punctured it. The material composition of septum may preferably be silicone. Alternatively, the material composition of the septum may also be a blend of different materials including but not limited to bromobutyl, chlorobutyl, isoprene, polyisoprene, SBR, polybudtadiene, EPDM, natural rubber and silicone. Alternatively, the fluid pathway including the dispense port could be a rigid plastic with a silicone injected overmold to produce the septum.

In some embodiments, the patient wears the automated injection device. In some embodiments, the automated injection device adheres to the patient's skin. In some embodiments, the automated injection device is attached to the patient during use via an adhesive. In some embodiments, the adhesive is disposed on the housing of the injection device. For example, referring to FIG. 1, the underside of the lower housing (306) of the injection device (300) may carry an adhesive for securing the injection device (300) temporarily to the skin of a patient until the injection is complete.

After the injection device is placed against or adhered to the skin, a safety mechanism or lock-out mechanism may be automatically released to allow the injection device to fire (inject). In other words, the injection device may be prevented from being actuated (it is locked out) until it is placed against the skin. Alternatively, the user/patient may manually remove a safety mechanism such as a safety pin, safety sleeve, or collar to release the injection device so that it is ready to fire (inject). The injection device preferably cannot be fired until the safety mechanism is released. The safety mechanism may be passive or active, and manually triggered by the user/patient, or automatically triggered by the injection device.

In other aspects, the instant injection device includes a mandrel within the expandable member to pre-stress the expandable member to a slightly expanded position when unfilled, so that when the expandable member expels the high concentration defibrotide formulation, it will contract or collapse to a condition where it is still stretched or stressed and continues to exert pressure on any defibrotide formulation contained within it. For example, referring to FIG. 2B, a mandrel (328) may be disposed within the expandable member (312) to pre-stress the expandable member (312) to a slightly expanded configuration when unfilled. This better assures that all or substantially all of the defibrotide formulation is fully expelled from the injection device (300). The mandrel (328) could be a fluid filled expandable member if desired. This would allow for a variable size mandrel. Alternatively, the expandable member could have a sufficiently small internal volume (small diameter) when unstressed so that virtually all defibrotide formulation is expelled without the need for an internal mandrel. Additionally, the expandable member could be flattened/stretched by ‘wrapping’ it around a surface within the injection device such as a cylindrical wall. The pre-stress created in the expandable member would act to eliminate any residual fluid volume remaining within it.

In use, the defibrotide formulations described here are delivered into the expandable member by the transfer apparatus and the expandable member expanded to a certain outer diameter, which can be controlled by the configuration of the inner surfaces of the housing. In this way, the entire length of the expandable member can be filled with a known volume of the defibrotide formulation since the outer diameter is known at each lengthwise location along the expandable member. In some embodiments, it is desirable to have the expandable member fill and empty along its length in a controlled way, from one end to the other to encourage the expandable member to completely empty, and to allow the easy and accurate measurement of the defibrotide formulation in the expandable member. To visually aid in determining how much of the defibrotide formulation is in the expandable member, graduated markings could be printed on the expandable member, like a syringe, to indicate the volume remaining in the expandable member. The expandable member and housing could be clear to allow the user to see the drug and the volume remaining in the injection device. Alternatively, graduated markings could be printed on the housing to indicate the volume remaining in the expandable member.

Again referring to FIGS. 1 and 2A, the high concentration low viscosity defibrotide formulation may be expelled progressively from the distal end (320) of the elongated expandable member (312) toward the proximal end (318). The proximal end (318) of the expandable member (312) is closest to the cannula of the injection device (300). This allows the user/patient to visually ascertain or approximate the injection status visually alone or with the aid of graduation markings on the injection housing, the window or the expandable member. Progressive expulsion may be achieved in a variety of ways. For example, the defibrotide formulation may exit the expandable member (312) at the manifold (326) at the proximal exit port section and is preferably located at the proximal end (318) of the elongated expandable member (312). The thickness of the wall of the expandable member (312) may be varied, uniformly or stepwise increased, along its length from the distal end (320) toward the proximal end (318). Due to restraint by the walls of the spiral channel (314) in which the expandable member (312) resides, the expandable member (312) may be inflated with the defibrotide formulation to a substantially uniform diameter along its length. However, the thicker wall (324) at the distal end (320) of the expandable member (312) may exert greater contraction force on the defibrotide formulation than the thinner wall at the proximal end (318) and thus collapse or contract in diameter first during expulsion of the defibrotide formulation. The expandable member may then collapse progressively from the distal end (320) toward the proximal end (318) as the wall (322) of the expandable member becomes thinner along its length in that direction.

Because the thickness of the expandable member preferably substantially uniformly increases from the proximal end toward the distal or closed end, the contractive force of the expandable member wall when expanded may increase substantially uniformly along the length of the elongated expandable member from the proximal port end to the distal or closed end. Thus, when the defibrotide is expelled into the patient, the expandable member may progressively collapse in diameter as well as shrink in length, which collapse in diameter and shrinkage in length is preferably viewable by the user/patient as described above. The distal end of the elongated expandable member may further allow for the connection of a movable indicator component in the injection device which will follow the shrinkage in length of the elongated expandable member. This indicator is preferably viewable by the user through the outer housing and indicates the state of the injection device and the progress of the injection. Alternatively, the expandable member is configured with a constant wall thickness and could be pre-stressed in manufacturing to bias it to fill from the proximal end to the distal end and collapse or empty from the distal end to the proximal end in a progressive manner as previously discussed.

The elongated expandable member of the injection device may be configured to have a section of the expandable member adjacent to the proximal exit port end that fills first and collapses last during filling and expulsion of the defibrotide formulation from the injection device. In other words, during filling of the injection device by the transfer apparatus, it is advantageous to have the most proximal exit port section of the expandable member to fill with the defibrotide formulation first. Additionally, during dispense of the defibrotide formulation from the injection device, it is advantageous to have the last remaining volume of the defibrotide formulation to be contained within the most proximal exit port section of the expandable member. There are several advantages to the abovementioned configuration. The proximal end section of the expandable member could have a thin wall that would cause it to remain inflated under a lower pressure than the rest of the expandable member. This would assure that the section of the expandable member would remain inflated until all defibrotide had been expelled from the rest of the expandable member. This section may be directly coupled to an empty indicator to provide for full or empty indication. Additionally, this section could be mechanically coupled to the empty indicator to allow for the automatic withdrawal of the button and cannula upon complete expulsion of the defibrotide formulation.

Alternatively, or in addition to varying the wall thickness of the expandable member, an elongated internal mandrel or shaft within the expandable member may progressively (linearly or stepwise) decrease in cross-sectional size along the length of the expandable member from proximal end (the exit port end) toward the distal end (closed end) of the expandable member. Additionally, the manifold, which allows for attachment of the expandable member to the injection device, may also be configured with a large diameter section at the proximal end of the expandable member. A large diameter section of the mandrel or manifold at the proximal end exit port of the expandable member may insure that the expandable member will fill with the defibrotide formulation in this area first. The expandable member may be held at nearly a fill diameter at the proximal end exit port by the large diameter section of the mandrel or manifold. As the defibrotide formulation first starts to fill the expandable member, it reaches a fill diameter first in the large diameter section then fills progressively along the length of the expandable member from the proximal end to the distal end as previously discussed.

During dispense of defibrotide formulation from the expandable member, the diameter of the expandable member at its distal end may continuously collapse in a progressive fashion (similar to deflating a long skinny balloon) from its distal to proximal end until all of the defibrotide formulation is expelled from the expandable member. A large diameter section of the mandrel or manifold at the proximal end exit port of the expandable member provides the same benefit (as previously described for filling) during dispense of the defibrotide formulation. This large diameter section insures that the last remaining amounts of the defibrotide formulation in the expandable member will be contained and dispensed from this area. This section may be directly coupled to an empty indicator to provide for full or empty indication as well as for the automatic withdrawal of the button and cannula upon complete expulsion of the defibrotide formulation.

When performing self-injections with automated injection devices, protecting the patient from accidental needle sticks is generally a beneficial feature of the device. Typically, the needle is retracted within the device before and after use, preventing the user from accessing the needle. However, during the injection, the needle is extended outside of the device. If the automated injection device were body worn and inadvertently fell off the user during the injection, the needle would be exposed creating a potential needle stick hazard to the user. Accordingly, in some embodiments, the automated injection devices include a skin dislodgement sensor that automatically retracts the cannula if the injection device becomes dislodged from the skin during injection.

Furthermore, when performing self-injections with a syringe and needle, patients may have the need to temporarily stop or pause the injection due to acute pain or irritation at the injection site. This pause in flow of defibrotide into the injection site, accomplished by removing pressure on the plunger rod of the syringe, helps to reduce the pain at the injection site by allowing the defibrotide fluid bolus more time to diffuse into the surrounding tissue and thus reducing the local pressure and associated pain and irritation. However, as more drugs are being presented in automated injection devices, the ability to manually pause these types of automatic systems does not exist. Once an automated injection device is placed on the skin and the cannula is introduced, it is difficult for the user to pause the injection due to pain or irritation at the injection site. Accordingly, in some embodiments, the automated injection devices comprise an actuator (e.g., a button) capable of moving the cannula from a pre-dispense or first depth position to a dispense or second depth position, which in turn pauses the flow of the defibrotide formulation into the cannula and then into the skin, as previously described

### EXAMPLES

### Example 1—Effect of Buffers on Formulation Viscosity

Increasing the defibrotide concentration in the formulation was shown to increase both viscosity and osmolality. It is important for pharmaceutical preparations for oral or parenteral administration to be of low-viscosity and/or isotonic. In order to identify buffers or excipients that may lower the viscosity and/or osmolality of defibrotide formulations, a wide-panel screening of various buffers and excipients (including GRAS excipients) was performed using a 200 mg/mL defibrotide formulation.

Test formulations were prepared to target 200 mg/mL as shown in Table 2 below.

TABLE 2
Defibrotide Formulations using Various Buffers and Excipients

| | Defibrotide | | Average | | Shear |
|---|---|---|---|---|---|
| | Concentration | Osmolality | Viscosity | Viscosity | Rate |
| Formulation | (mg/mL) | (mmol/kg) | (cP) | (cP) | (s⁻¹) |
| Defitelio DP, 34 | 80 | Not Tested | 4.2 | 4.21 | 113 |
| mM Sodium | | | | 4.24 | 225 |
| Citrate, pH 7.3 | | | | 4.23 | 338 |
| | | | | 4.28 | 525 |
| 34 mM Sodium | 200 | 438 | 29.6 | 29.6 | 15 |
| Citrate, pH 7.3 | | | | 29.4 | 30 |
| | | | | 29.8 | 48.8 |
| | | | | 29.7 | 71.3 |
| 34 mM Sodium | | 544 | 58 | 61.9 | 7.5 |
| Citrate, pH 6.5 | | | | 57.9 | 22.5 |
| | | | | 56.5 | 30 |
| | | | | 55.5 | 37.5 |
| 34 mM Sodium | | 636 | 51.9 | 51.8 | 7.5 |
| Citrate, 100 mM | | | | 51.8 | 22.5 |
| NaCl, pH 7.3 | | | | 52 | 30 |
| | | | | 51.9 | 37.5 |
| 34 mM Sodium | | 643 | 46.1 | 46.6 | 3.45 |
| Citrate, 100 mM | | | | 45.5 | 15 |
| Arginine, pH 7.3 | | | | 46 | 30 |
| | | | | 46.2 | 45 |
| 34 mM Sodium | | 542 | 53.6 | 52.9 | 11.3 |
| Citrate, 0.1% | | | | 53.7 | 22.5 |
| PS-80, pH 7.3 | | | | 53.9 | 33.8 |
| | | | | 53.8 | 38.3 |
| 34 mM Sodium | | 994 | 38.3 | 38.3 | 15 |
| Citrate, 250 mM | | | | 38.2 | 30 |
| Lidocaine HCl, | | | | 38.4 | 37.5 |
| pH 7.0 | | | | 38.4 | 52.5 |
| 34 mM Sodium | | 435 | 46.4 | 46.1 | 15 |
| Citrate, pH 8.0 | | | | 46.3 | 22.4 |
| | | | | 46.4 | 30 |
| | | | | 46.6 | 45 |
| 34 mM Gly-Gly, | | 566 | 38.5 | 38.6 | 15 |
| 100 mM NaCl, | | | | 38.4 | 30 |
| pH 7.5 | | | | 38.5 | 37.5 |
| | | | | 38.6 | 54 |
| 34 mM Gly-Gly, | 200 | 560 | 39.7 | 39.4 | 15 |
| 100 mM | | | | 39.5 | 30 |
| Arginine, pH 7.5 | | | | 39.7 | 45 |
| | | | | 40.3 | 52.5 |
| 34 mM Gly-Gly, | | 359 | 38.2 | 37.9 | 15 |
| 0.1% PS-80, | | | | 38.2 | 30 |
| pH 7.5 | | | | 38.2 | 45 |
| | | | | 38.4 | 54 |
| 34 mM Gly-Gly, | | 950 | 34.1 | 34 | 15 |
| 250 mM | | | | 34.1 | 37.5 |
| Lidocaine HCl, | | | | 34.2 | 48.8 |
| pH 7.0 | | | | 34.6 | 60 |
| 34 mM Gly-Gly, | | 323 | 27.7 | 27.9 | 16.5 |
| pH 7.5 | | | | 27.7 | 31.5 |
| | | | | 27.6 | 51 |
| | | | | 27.7 | 75 |
| 34 mM Gly-Gly, | | 370 | 36.9 | 36.5 | 15 |
| pH 8.0 | | | | 37 | 33.8 |
| | | | | 37 | 45 |
| | | | | 37.2 | 56.3 |
| 34 mM Gly-Gly, | | 375 | 34 | 33.7 | 15 |
| pH 8.5 | | | | 33.7 | 37.5 |
| | | | | 34.1 | 48.8 |
| | | | | 34.3 | 60.8 |
| 34 mM Tris, pH | | 394 | 39.4 | 39.1 | 15 |
| 7.5 | | | | 39.2 | 30 |
| | | | | 39.4 | 45 |
| | | | | 39.8 | 52.5 |
| 34 mM HEPES, | | 379 | 43.9 | 43.8 | 15 |
| pH 7.5 | | | | 43.8 | 30 |
| | | | | 43.7 | 37.5 |
| | | | | 44.2 | 46.5 |
| 34 mM His, pH | | 364 | 37 | 36.9 | 15 |
| 7.3 | | | | 36.9 | 30 |
| | | | | 37 | 45 |
| | | | | 37.3 | 56.3 |

### INCORPORATION BY REFERENCE

All patents and publications referenced herein are hereby incorporated by reference in their entireties, including the publications disclosed below.

The publications discussed herein are provided solely for their disclosure prior to the filing date of the present application. Nothing herein is to be construed as an admission that the present disclosure is not entitled to antedate such publication by virtue of prior disclosure.

This application incorporates by reference the following publications and applications in their entireties for all purposes: U.S. Pat. No. 9,925,333; PCT/US2018/045152; U.S. 62/540,657 filed Aug. 3, 2017.

What is claimed is:

1. A method of preparing and/or administering a high concentration defibrotide formulation to a patient via an automated injection device,
wherein the automated injection device includes
a) a housing defining a central portion, wherein the housing has a circular profile and includes a spiral recess;
b) a bladder positioned within the housing, the bladder including a first end, a second end, and a wall extending between the first end and the second end, the bladder being expandable, wherein the bladder wall includes an area of increased thickness along a side of the bladder wall, wherein the area of increased thickness deflects less than a remaining portion of the bladder wall during administration to affect the movement of the distal end of the bladder along an arcuate path; and
c) a cannula disposed within the central portion of the housing and configured to be movable between multiple positions, including a pre-dispense position where the cannula is not in fluid communication with the bladder, and a dispense position where the cannula is in fluid flow communication with the bladder;
wherein the high concentration defibrotide formulation comprises between 100 mg/mL to about 400 mg/mL of defibrotide, and a viscosity reducer at a concentration of between about 5 mM and about 100 mM, wherein the formulation has a viscosity between about 5 and about 70 cP when measured at a temperature of between 15° C. and 25° C., and an osmolality between about 240 mOsm/kg and about 1000 mOsm/kg, and is formulated for parenteral delivery to the patient; and wherein an inner diameter of the cannula ranges from about 0.08 mm to about 0.41 mm and further wherein the administering includes:
disposing the cannula in the dispense position;
introducing the high concentration defibrotide formulation under pressure to expand the bladder such that the second end of the bladder moves along an arcuate path around the central portion of the housing and an elastic force in the wall of the bladder expels the high concentration defibrotide formulation from the bladder and through the cannula at a flowrate between about 0.5 ml/hour to about 700 ml/hour.

2. The method of claim 1, wherein the cannula inner diameter ranges from about 0.1 mm to about 0.3 mm.

3. The method of claim 1, wherein the cannula flow rate is between about 10 ml/hour to about 200 ml/hour, or is between about 15 ml/hour to about 150 ml/hour.

4. The method of claim 1, wherein the viscosity reducer is selected from the group consisting of glycylglycine, glycine, a hyaluronidase, sodium citrate, sodium succinate, histidine, TRIS buffer, HEPES buffer, sodium chloride, arginine, lidocaine, benzyl alcohol, polysorbate-80, succinic acid, acetic acid, phosphoric acid, tartaric acid, amino acids, cyclodextrin and derivatives, Captsiol®, Polyvinylpyrrolidone (PVP), Kolloidon 12 PF, Kolloidon 17PF (BASF), Kolliphor HS 15 (BASF), MES buffer, Macrogol (15) hydroxystearate, polyethylene glycol (15)-hydroxystearate, polyoxyethylated 12-hydroxystearic acid, and Solutol HS 15.

5. The method of claim 1, wherein the high concentration defibrotide formulation further comprises a buffer or excipient so that the nucleic acid is in the form of an alkali metal salt.

6. The method of claim 5, wherein the buffer or excipient is selected from the group consisting of sodium citrate, sodium succinate, histidine (“HIS”), TRIS buffer, HEPES buffer, sodium chloride, succinic acid, acetic acid, phosphoric acid, tartaric acid, arginine, lidocaine, benzyl alcohol, salts (e.g calcium chloride or magnesium chloride; sodium or phosphate salts), amino acids, cyclodextrin and derivatives, Captsiol®, Polyvinylpyrrolidone (PVP), Kolloidon 12 PF, Kolloidon 17PF (BASF), Kolliphor HS 15 (BASF), MES buffer, Macrogol (15) hydroxystearate, polyethylene glycol (15)-hydroxystearate, polyoxyethylated 12-hydroxystearic acid, Solutol HS 15, and polysorbate-80.

7. The method of claim 6, wherein the buffer or excipient is sodium citrate, sodium succinate, or sodium chloride at a concentration of less than about 80 mM sodium salt.

8. The method of claim 5, wherein the buffer or excipient is sodium citrate at a concentration of between about 20 mM and about 34 mM.

9. The method of claim 6, wherein the viscosity of the high concentration defibrotide formulation decreases over time.

10. The method of claim 9, wherein the viscosity of the high concentration defibrotide formulation decreases during storage.

11. The method of claim 1, wherein the viscosity of the high concentration defibrotide formulation decreases under increasing shear, agitation, and/or pressure.

12. The method of claim 11, wherein said shear increases during administration.

13. The method of claim 12, wherein the shear increases during administration via a needle or device.

14. The method of claim 1, wherein the high concentration defibrotide formulation is formulated for subcutaneous delivery.

15. The method of claim 1, wherein the high concentration defibrotide formulation demonstrates extended systemic half-life compared to a defibrotide formulation not comprising a said viscosity reducer.

16. The method of claim 1, wherein the high concentration defibrotide formulation delivered subcutaneously with the device exhibits lower peak-to-trough ratios of plasma concentrations compared to a defibrotide formulation delivered intravenously.

17. The method of claim 1, wherein the high concentration defibrotide formulation exhibits improved efficacy and/or an improved safety profile when delivered subcutaneously with the device compared to a defibrotide formulation not comprising said viscosity reducer and delivered intravenously.

18. The method of claim 1, wherein the high concentration defibrotide formulation is isotonic or thixotropic.

19. The method of claim 1, wherein the high concentration defibrotide formulation comprises about 120-200 mg/mL of defibrotide and about 20 mM-60 mM viscosity reducer, and is formulated for subcutaneous delivery to a patient.

20. The method of claim 19, wherein the high concentration defibrotide formulation further comprises between about 10 mM to about 34 mM sodium citrate.

21. The method of claim 1, wherein the high concentration defibrotide formulation comprises about 120-200 mg/mL of defibrotide, about 20 mM-60 mM viscosity reducer, and about 10-25 mM sodium citrate, wherein the formulation is formulated for subcutaneous or intravenous delivery to a patient.

22. The method of claim 1, wherein the high concentration defibrotide formulation comprises about 120-200 mg/mL of defibrotide, about 20 mM-60 mM viscosity reducer, and about 25-35 mM sodium citrate, wherein the formulation is formulated for subcutaneous delivery to a patient.

23. The method of claim 1, wherein the high concentration defibrotide formulation is administered to treat or prevent a disease or condition selected from thrombosis, Hematopoietic Stem Cell Transplantation (HSCT) related complications including sinusoidal obstruction syndrome or hepatic veno-occlusive disease (VOD), Graft versus Host Disease (GvHD), Transplant-Associated Thrombotic Microangiopathy (TA-TMA) or Idiopathic Pneumonia Syndrome, other TMAs including Thrombotic Thrombocytopenic Purpura (TTP) and Hemolytic-Uremic Syndrome (HUS), Acute Myocardial Ischemia, Ischemic Stroke, Ischemia Reperfusion Injury (IRI, including Kidney IRI), treatment and prevention of cytokine release syndrome (CRS) or Chimeric Antigen Receptor (CAR)-T Cell Related Encephalopathy Syndrome (CRES) or CAR-T neurotoxicity, Acute Respiratory Distress Syndrome (ARDS), Sickle Cell Vaso-occlusive Crisis (VOC), Sickle Cell Related Acute Chest Syndrome, Disseminated Intravascular Coagulation (DIC), Sepsis, Renal Insufficiency, other Coronary or Peripheral Artery Diseases, Hematological Malignancies or Solid Tumors.

24. The method of claim 24, wherein the high concentration defibrotide formulation is administered at a dosing regimen that provides improved patient quality of life by requiring a reduced administration volume and/or allowing less-frequent administration and/or a shorter duration of administration and/or convenience of administration on an outpatient basis.

25. The method of claim 1, wherein the injection device further comprises a cannula actuator coupled to the cannula of the injection device for moving the cannula, wherein the high concentration defibrotide formulation is administered by movement of the cannula from the pre-dispense position to i) the first depth position; and ii) to the second depth position; and then 3) retention of the injection cannula at the second depth position.

26. The method of claim 1, wherein the injection device further comprises a manifold disposed within the housing and coupled to the first end of the bladder, the manifold including a fluid inlet port configured to receive the high concentration defibrotide formulation and a fluid pathway configured for fluid communication between the fluid inlet port and the bladder for introduction of the high concentration defibrotide formulation under pressure into the bladder.

27. The method of claim 1, wherein the injection device further comprises an indicator disposed on the second end of the bladder and the method further comprises the step of providing an indication of defibrotide dosing progress to the patient via the indicator.

28. The method of claim 1, wherein the injection device further comprises a mandrel coupled to the second end of the bladder and the method further comprises the step of pre-stressesing the bladder to an expanded condition using the mandrel.

29. The method of claim 1, wherein the injection device comprises an adhesive disposed on the housing and the method further comprises the step of attaching the injection device to the patient using the adhesive for the duration of the treatment.

30. An automated injection device for delivery of a high concentration defibrotide formulation, wherein the automated injection device includes
a) a housing defining a central portion, wherein the housing has a circular profile and includes a spiral recess within which a second end of a bladder is positioned and moves along upon contraction and expansion;
b) a bladder positioned within the housing, the bladder including a first end, a second end, and a wall extending between the first end and the second end, the bladder being expandable, wherein the bladder wall includes an area of increased thickness along a side of the bladder wall, where the area of increased thickness deflects less than a remaining portion of the bladder wall during administration to affect the movement of the distal end of the bladder along an arcuate path;
c) a cannula disposed within the central portion of the housing and configured to be movable between multiple positions, including a pre-dispense position where the cannula is not in fluid communication with the bladder, and a dispense position where the cannula is in fluid flow communication with the bladder,
wherein the bladder comprises a fluid formulation of a high concentration defibrotide formulation comprising between 100 mg/mL to about 400 mg/mL of defibrotide, and a viscosity reducer at a concentration of between about 5 mM and about 100 mM, wherein the formulation has a viscosity between about 5 and about 70 cP when measured at a temperature of between 15° C. and 25° C., and an osmolality between about 240 mOsm/kg and about 1000 mOsm/kg, and is formulated for parenteral delivery to the patient and wherein the inner diameter of the cannula ranges from about 0.08 mm to about 0.41 mm to result in a flowrate between about 0.5 ml/hour to about 700 ml/hour.

31. The automated injection device of claim 30, wherein the cannula inner diameter ranges from about 0.1 mm to about 0.3 mm.

32. The automated injection device of claim 30, wherein the cannula includes a side hole configured so that when the cannula is in the pre-dispense position or in a first depth position with respect to a surface of a patient's skin, the side hole is not in fluid communication with the bladder, and when the cannula is in the dispense position or a second depth position with respect to the surface of the patient's skin, the side hole is in fluid flow communication with the bladder, the second depth position being closer to the patient's skin surface than the first depth position.

33. The automated injection device of claim 32, further comprising a septum positioned within the housing and configured to seal around a periphery of the side hole of the cannula when the cannula is in the pre-dispense position or in the first depth position.

34. The automated injection device of claim 30, further comprising a cannula actuator coupled to the cannula of the injection device for moving the cannula, wherein the high concentration defibrotide formulation is administered by movement of the cannula from the pre-dispense position to i) the first depth position; and ii) to the second depth position; and then 3) retention of the injection cannula at the second depth position.

35. The automated injection device of claim 30, further comprising a manifold disposed within the housing and coupled to the first end of the bladder, the manifold including a fluid inlet port configured to receive the high concentration defibrotide formulation and a fluid pathway configured for fluid communication between the fluid inlet port and the bladder for introduction of the high concentration defibrotide formulation under pressure into the bladder.

36. The automated injection device of claim 30, further comprising an indicator disposed on the second end of the bladder that provides an indication of dosing progress to the patient.

37. The automated injection device of claim 30, further comprising a mandrel coupled to the second end of the bladder that pre-stresses the bladder to an expanded condition.

38. The automated injection device of claim 30, further comprising an adhesive disposed on the housing for attaching the injection device to the patient.

39. A method to prepare a high concentration defibrotide formulation using an automated injection device of any one of claims 30-38.
